(19) United States
(12) Patent Application Publication
(10) Pub. No.: US 2023/0096663 A1
(43) Pub. Date: Mar. 30, 2023

(54) FARNESOID X RECEPTOR MODULATING COMPOUNDS AND METHODS OF USING THE SAME

(71) Applicant: Inorbit Therapeutics AB, Billdal (SE)

(72) Inventors: Robert Judkins, Mölndal (SE); Lambertus Benthem, Billdal (SE); Rajiv Sharma, Fremont, CA (US)

(21) Appl. No.: 17/929,006

(22) PCT Filed: Feb. 11, 2021
(86) PCT No.: PCT/IB2021/051138

§ 371 Date: Aug. 10, 2022

Related U.S. Application Data
(60) Provisional application No. 62/972,782, filed on Feb. 11, 2020.

(51) Int. Cl. A61K 31/55; A61P 1/16

(52) U.S. Cl. CPC A61K 31/55; A61P 1/16

(57) ABSTRACT

Provided are compounds that can act as a modulator of a farnesoid X receptor (FXR) and that can be useful in the treatment of diseases and/or disorders associated with the FXR. Compositions including such compounds are also provided along with methods for preparing compounds of the present invention and their use.

### CROSS-REFERENCE TO RELATED APPLICATIONS

This application claims the benefit of U.S. Provisional Patent Application Ser. No. 62/972,782, filed Feb. 11, 2020, the disclosure of which is incorporated herein by reference in its entirety.

### FIELD

The present invention relates to compounds that can act as modulators of a farnesoid X receptor (FXR) and that can be useful in the treatment of diseases and/or disorders associated with the FXR. In some embodiments, the present invention relates to compounds and compositions that modulate a FXR and methods for their preparation and use.

### BACKGROUND

The farnesoid X receptor is a member of the nuclear hormone receptor superfamily. The FXR functions as a heterodimer with the retinoid X receptor (RXR) and binds to response elements in the promoter region of target genes to regulate gene transcription.

Studies have shown that FXR plays an important role in control of enterohepatic circulation of bile acids, bile acid synthesis, and secretion and bile acid uptake into hepatocytes. This aspect has been exploited by many researchers for finding appropriate drug targets for the treatment of NASH/NAFLD.

The farnesoid X receptor is an orphan nuclear receptor initially identified from a rat liver cDNA library (BM. Forman, et al., Cell, 1995, 81(5), 687-693) that is most closely related to the insect ecdysone receptor. FXR is a member of the nuclear receptor superfamily of ligand-activated transcription factors that includes receptors for the steroid, retinoid, and thyroid hormones (DJ. Mangelsdorf, et al., Cell, 1995, 83(6), 841-850). FXR is expressed in various tissues including liver, kidney, intestine, colon, ovary, and adrenal gland (see Forman et al, Cell 81:687-693, 1995; Lu et al J. Biol. Chem., 17:17, 2001) and is a key regulator of cholesterol homeostasis, triglyceride synthesis and lipogenesis (Crawley, Expert Opinion Ther. Patents (2010), 20(8): 1047-1057).

The relevant physiological ligands of FXR are bile acids (D. Parks et al., Science, 1999, 284(5418), 1362-1365). The most potent one is chenodeoxycholic acid (CDCA), which regulates the expression of several genes that participate in bile acid homeostasis. Farnesol and derivatives, together called farnesoids, are originally described to activate the rat orthologue at high concentration but they do not activate the human or mouse receptor. Beyond controlling intracellular gene expression, FXR seems to also be involved in paracrine and endocrine signaling by upregulating the expression of the cytokine Fibroblast Growth Factor (J. Holt et al., Genes Dev., 2003, 17(13), 1581-1591; T. Inagaki et al., Cell Metab., 2005, 2(4), 217-225).

Activation of the FXR has the potential to be a treatment for a range of diseases including bile acid related disorders, metabolic syndrome, type-2-diabetes, hyperlipidemia, hypertriglyceridemia, primary biliary cirrhosis (PBC), fatty liver disease, nonalcoholic steatohepatitis (NASH), inflammatory autoimmune diseases, Crohn's disease, multiple sclerosis, atherosclerosis, kidney disorders (including chronic kidney disease), hepatic and colon cancers, and other disorders. Although numerous FXR modulators are known and have been disclosed (for recent examples see, A. Zampella et al., Expert Opinion Ther. Patents (2018), 28(5): 351-364), there is still a need for the development of novel and potent compounds for the treatment and prevention of disease.

The listing or discussion of an apparently prior-published document in this specification should not necessarily be taken as an acknowledgement that the document is part of the state of the art or is common general knowledge.

### SUMMARY

One aspect of the present invention is directed to compounds represented by Formula I, or an enantiomer, stereoisomer, tautomer, solvate, hydrate, prodrug, amino acid conjugate, metabolite, or pharmaceutically acceptable salts thereof:

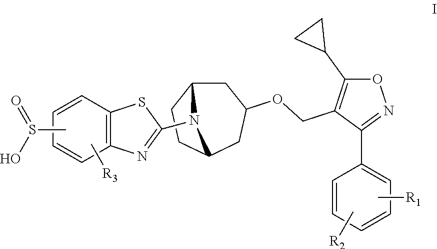

wherein:

R₁ and R₂ are independently selected from the group consisting of hydrogen, chloro, methyl, trifluoromethyl, methoxy, trifluromethoxy and cyclopropyl;

R₃ is hydrogen, chloro, methyl, methoxy, fluoro or trifluoromethoxy.

In some aspects, the invention provides a compound of Formula II:

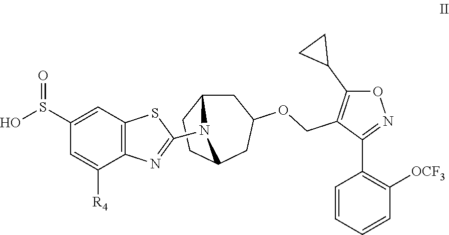

wherein:

R₄ is fluoro or methyl;

or an enantiomer, stereoisomer, tautomer, solvate, hydrate, prodrug, amino acid conjugate, metabolite, or pharmaceutically acceptable salt thereof.

In some embodiments, the invention provides a compound selected from the group consisting of sodium 2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-sulfinatea and sodium 2-((1R,3r,5S)-3-((5-cyclopropyl-3-(2-(trifluoromethoxy)phenyl)isoxazol-4-yl)methoxy)-8-azabicyclo[3.2.1]octan-8-yl)-4-methylbenzo[d]thiazole-6-sulfinate.

Another aspect of the present invention is directed to a method of modulating a FXR. The method comprises administering to a subject in need thereof an effective amount of a compound of the present invention (e.g., a compound of Formula I-II or a pharmaceutically acceptable salt, hydrate, solvate, prodrug, stereoisomer, or tautomer thereof).

Another aspect of the present invention is directed to a method of activating a FXR. The method comprises administering to a subject in need thereof an effective amount of a compound of the present invention (e.g., a compound of Formula I-II or a pharmaceutically acceptable salt, hydrate, solvate, prodrug, stereoisomer, or tautomer thereof).

Another aspect of the present invention is directed to a method of treating and/or preventing a disease and/or disorder. In some embodiments, the disease and/or disorder is a bile acid related disorder, metabolic syndrome, type-2 diabetes, diabetic nephropathy, hyperlipidemia, hypertriglyceridemia, obesity, liver cirrhosis, liver fibrosis, primary biliary cirrhosis (PBC), primary sclerosing cholangitis (PSC), fatty liver disease, nonalcoholic steatohepatitis (NASH), nonalcoholic fatty liver disease (NAFLD), alcoholic liver disease, chemotherapy associated steatohepatitis (CASH), hepatitis B, inflammatory autoimmune diseases, inflammatory bowel disease, Crohn's disease, ulcerative colitis, proctitis, pouchitis, Celiac's Disease, bile acid diarrhea, multiple sclerosis, atherosclerosis, kidney disorders (including chronic kidney disease), kidney fibrosis, lung fibrosis, cancer including hepatic cancers, colon cancers and breast cancers, and other disorders. The method comprises administering to a subject in need thereof a therapeutically effective amount of a compound of the present invention (e.g., a compound of Formula I-II or a pharmaceutically acceptable salt, hydrate, solvate, prodrug, stereoisomer, or tautomer thereof)) or a pharmaceutical composition comprising said compound of the present invention.

A further aspect of the present invention is directed to a pharmaceutical composition comprising a compound of the present invention (e.g., a compound of Formula I-II or a pharmaceutically acceptable salt, hydrate, solvate, prodrug, stereoisomer, or tautomer thereof) and a pharmaceutically acceptable carrier. The pharmaceutical composition may further include an excipient, diluent, and/or surfactant.

Another aspect of the present invention relates to a compound of the present invention (e.g., a compound of Formula I-II or a pharmaceutically acceptable salt, hydrate, solvate, prodrug, stereoisomer, or tautomer thereof)) for use in the manufacture of a medicament for treating and/or preventing a disease and/or disorder in which a farnesoid X receptor (FXR) plays a role. In some embodiments, a FXR plays a role in a disease and/or disorder in that the FXR is involved in a pathway, mechanism, or action associated with the disease and/or disorder such as, e.g., in the control of enterohepatic circulation of bile acids, bile acid synthesis, and/or secretion and bile acid uptake into hepatocytes.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWING

FIG. 1 is a graph showing the fold change of BSEP gene expression after incubation of the compound of Example 2 with primary human hepatocytes.

FIG. 2 is a graph showing the fold change of SHP gene expression after incubation of the compound of Example 2 with primary human hepatocytes.

FIG. 3 is a graph showing the fold change of BSEP gene expression after incubation of the compound of Example 3 with primary human hepatocytes.

FIG. 4 is a graph showing the fold change of SHP gene expression after incubation of the compound of Example 3 with primary human hepatocytes.

FIG. 5 is a graph showing liver triglyceride content in HFHC male mice after 28 days of treatment with the compound of Example 2.

FIG. 6 is a graph showing liver cholesterol content in HFHC male mice after 28 days of treatment with the compound of Example 2.

FIG. 7 is a graph showing plasma total cholesterol content in HFHC male mice after 28 days of treatment with the compound of Example 2.

FIG. 8 is a graph showing liver triglyceride content in HFHC male mice after 28 days of treatment with the compound of Example 3.

FIG. 9 is a graph showing liver cholesterol content in HFHC male mice after 28 days of treatment with the compound of Example 3.

FIG. 10 is a graph showing plasma total cholesterol content in HFHC male mice after 28 days of treatment with the compound of Example 3.

FIG. 11 illustrates how left lateral lobes of livers were separated and dissected according to a method described herein.

FIG. 12 shows the NAFLD Activity score in a STAM model when compound of example 2 is administered once daily at a 0.3 mg/kg, 1 mg/kg, or 3 mg/kg dose for 3 weeks.

FIG. 13 shows the Sirius Red Positive area (%) in a STAM model when compound of Example 2 is administered once daily at a 0.3 mg/kg, 1 mg/kg, or 3 mg/kg dose for 3 weeks.

FIG. 14 is a MS/MS spectrum of metabolites of the compound of Example 2.

### DETAILED DESCRIPTION OF EXAMPLE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”). Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, the terms “increase,” “increases,” “increased,” “increasing,” and similar terms indicate an elevation in the specified parameter or value of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more.

As used herein, the terms “reduce,” “reduces,” “reduced,” “reduction,” “inhibit,” and similar terms refer to a decrease in the specified parameter or value of at least about 5%, 10%, 15%, 20%, 25%, 30%, 35%, 40%, 45%, 50%, 55%, 60%, 65%, 70%, 75%, 80%, 85%, 90%, 95%, 97%, or 100%.

A “farnesoid X receptor” or “FXR” as used herein is a farnesoid X receptor from any source and/or that is present in a subject and/or expressed in any form. In some embodiments, a farnesoid X receptor is from and/or is present and/or expressed in an animal such as, e.g., a mammal. In some embodiments, a farnesoid X receptor is from and/or is present and/or expressed in a primate, cow, sheep, goat, horse, dog, cat, rabbit, rat, mouse, fish, bird, and/or the like. In some embodiments, a farnesoid X receptor is from and/or is present and/or expressed in a human.

The terms “modulate” and “modulating”, in reference to a FXR, refer to the ability of a compound (e.g., a compound of the present invention) to activate or inhibit one or more function(s), action(s), and/or characteristic(s) of the FXR, either directly or indirectly. This may occur in vitro or in vivo and is intended to encompass antagonism, agonism, partial antagonism and/or partial agonism of a function, action, and/or characteristic associated with a FXR.

The term “activating”, in reference to a FXR, refers to the ability of a compound (e.g., a compound of the present invention) to activate, increase or enhance a function, action, and/or characteristic associated with the FXR, and, thus, the compound is an FXR agonist.

The term “modulator”, in reference to a FXR, refers to a compound (e.g., a compound of the present invention) that modulates a FXR. In some embodiments, a compound of the present invention modulates a FXR by activating one or more function(s), action(s), and/or characteristic(s) of the FXR.

The term “agonist” refers to a compound (e.g., a compound of the present invention) that combines with and/or binds to a specific receptor (e.g., a FXR) and activates, increases or enhances a function, action, and/or characteristic associated with the receptor. The ern “agonist” includes both a full agonist and a partial agonist, which activates, increases or enhances a function, action, and/or characteristic associated with the receptor (e.g., FXR) to a lesser extent than a full agonist and/or has partial efficacy at the receptor compared to a full agonist. In some embodiments, a compound of the present invention is an FXR agonist. In some embodiments, a compound of the present invention is an agonist and activates a FXR providing the same or substantially the same reaction and/or pharmacological response typically produced by the binding of an endogenous agonist.

“Substantially the same” as used herein in reference to a measurable value and/or response means being within about ±10% of the compared to value and/or response.

The term “sulfinic acid” means the functional group —S(O)OH, consisting of a sulfinyl group and a hydroxyl group.

The term “sulfinate” means the conjugate base of sulfinic acid, where the hydroxyl has been deprotonated to give S(O)O⁻.

The term “sulfonic acid” means the functional group —S(O)₂OH, consisting of a sulfonyl group and a hydroxyl group.

The term “optionally substituted” is understood to mean that a given chemical moiety (e.g., an alkyl group) can (but is not required to) be bonded to other substituents (e.g., heteroatoms). For instance, an alkyl group that is optionally substituted can be a fully saturated alkyl chain (e.g., a pure hydrocarbon). Alternatively, the same optionally substituted alkyl group can have one or more substituent(s) different from hydrogen. For instance, it can, at any point along the chain, be bound to a halogen atom, a hydroxyl group, or any other substituent described herein. Thus, the term “optionally substituted” means that a given chemical moiety has the potential to contain other functional groups, but does not necessarily have any further functional groups.

The term “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposable mirror images of one another.

The term “pharmaceutically acceptable salt” refers to a salt of a compound which is, within the scope of sound medical judgment, suitable for use in contact with the tissues of humans and lower animals without undue toxicity, irritation, allergic response and the like, and is commensurate with a reasonable benefit/risk ratio. Pharmaceutically acceptable salts are well known in the art.

For a detailed review of pharmaceutically acceptable salts see J. Pharmaceutical Sciences, 66: 1-19 (1977), by Berge et al. In some embodiments, the salts can be prepared in situ during the final isolation and/or purification for a compound of the invention, or separately by reaction of the free acid function with a suitable inorganic or organic base. Suitable salts include, but are not limited to, metals, such as sodium, potassium and calcium, or amines, such as triethylammonium, ethanolammonium and lysine.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “prodrug” refers to a prodrug of a compound which is, within the scope of sound medical judgment, suitable for use in contact with the tissues of humans and lower animals with undue toxicity, irritation, allergic response, and/or the like, commensurate with a reasonable benefit/risk ratio, and effective for their intended use, as well as the zwitterionic forms, where possible, of a compound of the present invention. “Prodrug”, as used herein means a compound that is convertible in vivo by metabolic means (e.g., by hydrolysis) to afford a compound of the present invention (e.g., a compound of Formula I-II). Various forms of prodrugs are known in the art, for example, as discussed in Bundgaard, (ed.), Design of Prodrugs, Elsevier (1985); Widder, et al. (ed.), Methods in Enzymology, Vol. 4, Academic Press (1985); Krogsgaard-Larsen, et al., (ed). “Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

The term “amino acid conjugate” refers to a conjugate of a compound of the present invention (e.g., a compound of Formula I-II) with an amino acid. Preferably, such amino acid conjugates of the present invention will have the added advantage of enhanced integrity in bile and/or intestinal fluids. Suitable amino acids include, but are not limited to, glycine and taurine. Thus, the present invention encompasses the glycine and taurine conjugates of a compound of Formula I-II.

The term “GW4064” is an FXR agonist compound having the following structure:

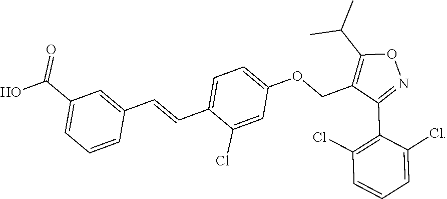 [SMILES: CC(C)c1onc(-c2c(Cl)cccc2Cl)c1COc1ccc(/C=C/c2cccc(C(=O)O)c2)c(Cl)c1]

Unless otherwise stated, structures depicted herein are meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Provided according to embodiments of the present invention are compounds having a structure of Formula I:

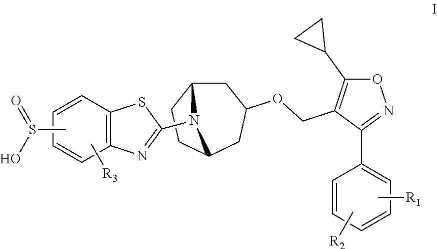

wherein:

R₁ and R₂ are independently selected from the group consisting of hydrogen, chloro, methyl, trifluoromethyl, methoxy, trifluromethoxy and cyclopropyl; and

R₃ is hydrogen, chloro, methyl, methoxy, fluoro or trifluoromethoxy.

In some embodiments, the invention provides a compound having a structure of Formula II

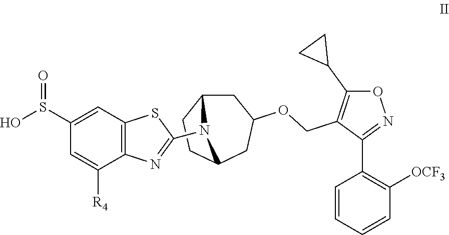

wherein:

R₄ is fluoro or methyl;

or an enantiomer, stereoisomer, tautomer, solvate, hydrate, prodrug, amino acid conjugate, metabolite, or pharmaceutically acceptable salt thereof.

Some embodiments of the present invention provide a compound selected from:
sodium 2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-sulfinate and sodium 2-((1R,3r,5S)-3-((5-cyclopropyl-3-(2-(trifluoromethoxy)phenyl)isoxazol-4-yl)methoxy)-8-azabicyclo[3.2.1]octan-8-yl)-4-methylbenzo[d]thiazole-6-sulfinate.

In some embodiments, a compound of the present invention is a metabolite (i.e. having undergone metabolism or biotransformation in the subject). In some embodiments, a compound of the present invention is a sulfinic acid (or its corresponding sulfinate salt) compound. In some embodiments, a compound of the present invention may be a sulfinic acid metabolite, which may be a corresponding sulfonic acid of the compound (e.g., a compound having a —S(O)₂OH or —S(O)₂O⁻ group replacing a —S(O)OH or —S(O)O⁻ group in the compound) or a corresponding sulfinate ester of the compound (e.g., a compound having a —S(O)O(C₁₋₆ alkyl) group replacing a —S(O)OH or —S(O)O⁻ group in the compound). In some embodiments, a compound of the present invention is a sodium salt. In some embodiments, a compound of the present invention is a sulfinate salt (e.g. a sodium sulfinate salt).

In some embodiments, a compound of the present invention may have a different metabolic profile in-vivo compared to a corresponding carboxylic acid compound (i.e., a compound having a —COOH or —COO⁻ group replacing a —S(O)OH or —S(O)O⁻ group in the compound). These corresponding carboxylic acid compound are typically metabolised to the acyl-glucuronide and such metabolism can give rise to reactive metabolites that cause liver toxicity and drug induced liver injury (Shipkova M, Armstrong V W, Oellerich M, and Wieland E (2003) Acyl glucuronide drug metabolites: Toxicological and analytical implications. Ther Drug Monit 25: 1-16; Regan S, Maggs J, Hammond T, Lambert C, Williams D and Park B K (2010) Acyl glucuronides: the good, the bad and the ugly. Biopharm Drug Dispos 31: 367-395; Shipkova M, Armstrong V W, Oellerich M, and Wieland E (2003) Acyl glucuronide drug metabolites: Toxicological and analytical implications. Ther Drug Monit 25: 1-16).

In some embodiments, a compound of the present invention may only be metabolised by oxidative pathways, such as Cyp oxidation, and/or may avoid acyl glucuronide like metabolites compared to a corresponding carboxylic acid compound.

A compound of the invention may break down in-vivo via a different metabolic pathway than a corresponding carboxylic acid compound and/or the compound of the invention may have beneficial liver safety effects and/or improved liver safety and/or improved efficacy compared to a corresponding carboxylic acid compound.

In some embodiments, a compound of the present invention may have beneficial liver safety effects and/or improved liver safety and/or improved efficacy compared to another compound such as, e.g., a corresponding carboxylic acid compound.

In some embodiments, a compound of the present invention may be more efficacious than, be less toxic than, be longer acting than, be more potent than, produce fewer side effects than, be more easily absorbed than, have a better pharmacokinetic profile (e.g., higher oral bioavailability and/or lower clearance) than, and/or have other useful pharmacological, physical and/or chemical properties than a compound known in the prior art. Such effects may be evaluated clinically, objectively and/or subjectively by a health care professional, a treatment subject or an observer.

In some embodiments, a compound of the present invention may have a different distribution profile when orally dosed in-vivo, such as increased exposure in the liver versus plasma, compared to a corresponding carboxylic acid compound.

In some embodiments, a compound of the present invention may upregulate and/or increase expression of a FXR related gene, optionally in a subject and/or in vitro. The FXR related gene may be upregulated and/or increased by at least about 5%, 10%, 15%, 20%, 25%, 30%, 35%, 40%, 45%, 50%, 55%, 60%, or more. In some embodiments, a compound of the present invention upregulates a BSEP gene and/or a SHP gene.

In some embodiments, a compound of the present invention may reduce expression of a liver enzyme, optionally in a subject and/or in vitro. Expression of a liver enzyme may be reduced by at least about 5%, 10%, 15%, 20%, 25%, 30%, 35%, 40%, 45%, 50%, 55%, 60%, or more. In some embodiments, the compound reduces expression of CYP7A1.

In some embodiments of the invention, a compound of the present invention may show high exposure in the liver vs the plasma when establishing distribution profiles.

In some embodiments of the invention, a compound of the present invention may reduce liver triglycerides in the liver and/or plasma. In some embodiments, a compound of the present invention may reduce liver triglycerides in the liver and/or plasma by at least about 5%, 10%, 15%, 20%, 25%, 30%, 35%, 40%, 45%, 50%, 55%, 60%, or more. In some embodiments of the invention, a compound of the present invention may reduce cholesterol in the liver and/or plasma. In some embodiments, a compound of the present invention may reduce cholesterol in the liver and/or plasma by at least about 5%, 10%, 15%, 20%, 25%, 30%, 35%, 40%, 45%, 50%, 55%, 60%, or more.

In some embodiments, a compound and/or method of the present invention may reduce steatosis, hepatocellular ballooning, and/or lobular inflammation in a subject that is administered the compound. In some embodiments, a compound and/or method of the present invention reduces steatosis, hepatocellular ballooning, and/or lobular inflammation in the subject by at least about 5%, 10%, 15%, 20%, 25%, 30%, 35%, 40%, 45%, 50%, 55%, 60%, 65%, 70%, 75%, 80%, 85%, 90%, 95%, 97%, or more compared to steatosis, hepatocellular ballooning, and/or lobular inflammation in the subject prior to administration of the compound and/or compared to a control. In some embodiments, a compound and/or method of the present invention may reduce a subject's NAFLD activity score (NAS) after administration of the compound. In some embodiments, a compound and/or method of the present invention may reduce a subject's NAFLD activity score (NAS) by at least about 5%, 10%, 15%, 20%, 25%, 30%, 35%, 40%, 45%, 50%, 55%, 60%, 65%, 70%, 75%, 80%, 85%, 90%, 95%, 97%, or more compared to the subject's NAS prior to administration of the compound and/or compared to a control.

In some embodiments, a compound and/or method of the present invention may reduce fibrosis in a subject that is administered the compound. In some embodiments, a compound and/or method of the present invention reduces fibrosis in the subject by at least about 5%, 10%, 15%, 20%, 25%, 30%, 35%, 40%, 45%, 50%, 55%, 60%, 65%, 70%, 75%, 80%, 85%, 90%, 95%, 97%, or more compared to fibrosis in the subject prior to administration of the compound and/or compared to a control.

In some embodiments, a compound of the present invention may be more efficacious than, be less toxic than, be longer acting than, be more potent than, produce fewer side effects than, be more easily absorbed than, have a better pharmacokinetic profile (e.g., higher oral bioavailability and/or lower clearance) than, and/or have other useful pharmacological, physical or chemical properties than a compound known in the prior art. Such effects may be evaluated clinically, objectively and/or subjectively by a health care professional, a treatment subject or an observer.

Provided according to some embodiments of the present invention is a composition (e.g., a pharmaceutical composition) comprising a compound of the present invention (e.g., a compound of Formula I-II). In some embodiments, a pharmaceutical composition of the present invention comprises a compound of the present invention and a pharmaceutically acceptable carrier.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient (e.g., a compound of the present invention), its use in the therapeutic and/or pharmaceutical compositions is contemplated.

Compounds of the invention are indicated as pharmaceuticals. According to a further aspect of the invention there is provided a compound of the invention, for use as a pharmaceutical (e.g. for use in medicine).

According to some embodiments, a compound and/or composition of the present invention is administered to a subject. In some embodiments, a method of modulating a farnesoid X receptor (FXR) in a subject is provided, the method comprising administering to the subject a compound of the present invention and/or a composition of the present invention. In some embodiments, a method of activating a farnesoid X receptor (FXR) is provided, the method comprising administering to a subject a compound of the present invention and/or a composition of the present invention.

In some embodiments, a method of treating and/or preventing a disease or disorder in which a farnesoid X receptor (FXR) plays a role is provided, the method comprising administering to a subject in need thereof an effective amount (e.g., a therapeutically effective amount, a treatment effective amount, and/or a prevention effective amount) of a compound of the present invention and/or a composition of the present invention. In some embodiments, the disease or disorder is bile acid related disorder, metabolic syndrome, type-2 diabetes, diabetic nephropathy, hyperlipidemia, hypertriglyceridemia, obesity, liver cirrhosis, liver fibrosis, primary biliary cirrhosis (PBC), primary sclerosing cholangitis (PSC), fatty liver disease, nonalcoholic steatohepatitis (NASH), nonalcoholic fatty liver disease (NAFLD), alcoholic liver disease, chemotherapy associated steatohepatitis (CASH), hepatitis B, inflammatory autoimmune diseases, inflammatory bowel disease, Crohn's disease, ulcerative colitis, proctitis, pouchitis, Celiac's Disease, bile acid diarrhea, multiple sclerosis, atherosclerosis, kidney disorders (including chronic kidney disease), kidney fibrosis, lung fibrosis, cancer including hepatic cancers, colon cancers and breast cancers, and other disorders.

The term “therapeutically effective amount” refers to an amount of a compound of the present invention (e.g., a compound of Formula I-II) that is sufficient to achieve or elicit a therapeutically useful response or a stated effect in a subject. Accordingly, a therapeutically effective amount of a compound of Formula I-II used for the treatment of a condition mediated by a FXR can be an amount sufficient for the treatment of the condition mediated by the FXR

As used herein, the term “subject” refers to an animal. Typically, the animal is a mammal. A subject also refers to, for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

The terms “treat”, “treating”, “treatment of” and grammatical variations thereof in reference to a disease, or condition refer to any type of treatment that imparts a benefit to a subject and may mean that the severity of the subject's condition is reduced, at least partially improved or ameliorated and/or that some alleviation, mitigation or decrease in at least one clinical symptom associated with a disease, disorder, or condition is achieved and/or there is a delay in the progression of the symptom. In some embodiments, the severity of a symptom associated with a disease, disorder, or condition mediated by a FXR may be reduced in a subject compared to the severity of the symptom in the absence of a method of the present invention. In some embodiments, “treat”, “treating”, “treatment of” and grammatical variations thereof in reference to a disease or disorder refer to ameliorating the disease or disorder (i.e., slowing or arresting or reducing the development of the disease or disorder or at least one clinical symptom thereof). In some embodiments, “treat”, “treating” or “treatment of” and grammatical variations thereof in reference to a disease or disorder refer to alleviating or ameliorating at least one physical parameter including those which may not be discernible by the subject. In some embodiments, “treat”, “treating” or “treatment of” and grammatical variations thereof in reference to a disease or disorder refer to modulating the disease or disorder, either physically (e.g., stabilization of a discernible symptom), physiologically (e.g., stabilization of a physical parameter), or both.

In some embodiments, a compound of the present invention may be administered to a subject in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a composition of the present invention.

The terms “prevent,” “preventing” and “prevention” (and grammatical variations thereof) refer to avoidance, reduction and/or delay of the onset of a symptom associated with a disease or disorder (e.g., a disease, disorder, or condition mediated by a FXR) and/or a reduction in the severity of the onset of symptom associated with a disease or disorder (e.g., a disease, disorder, or condition mediated by a FXR) relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention.

In some embodiments, a compound of the present invention may be administered in a prevention effective amount. A “prevention effective” amount as used herein is an amount that is sufficient to prevent (as defined herein) a symptom associated with a disease or disorder (e.g., a disease, disorder, or condition mediated by a FXR) in a subject. Those skilled in the art will appreciate that the level of prevention need not be complete, as long as some benefit is provided to the subject. In some embodiments, a prevention effective amount may be achieved by administering a composition of the present invention.

The terms “administer”, “administering”, “administration” and grammatical variations thereof as used herein refer to directly administering to a subject a compound of the present invention (or a pharmaceutically acceptable salt, etc., thereof) and/or a composition of the present invention. In some embodiments, a compound and/or composition of the present invention is administered to the subject in an amount that can form an equivalent amount of the active compound within the subject's body.

A compound of the present invention can be administered in a therapeutically effective amount to treat and/or prevent a disease or disorder and/or to prevent the development thereof in a subject. Administration of a compound of the present invention can be accomplished via any mode of administration for therapeutic agents such as, for example oral, rectal, topical, and/or parenteral administration may be employed. In some embodiments, a compound of the present invention is administered orally.

Depending on the intended mode of administration, a compound of the present invention and/or composition of the present invention can be in a dosage form known to those skilled in the pharmaceutical practices, such as, for example, injectables, tablets, suppositories, pills, time-release capsules, emulsions, syrups, powders, liquids, suspensions, and/or the like.

Typical pharmaceutical compositions include, but are not limited to, tablets, pills, powders or gelatin capsules comprising the active ingredient (e.g., a compound of the present invention) and a pharmaceutically acceptable carrier such as for example:

a) a diluent, e.g., purified water, corn oil, olive oil, sunflower oil, fish oils, such as EPA or DHA or their esters or triglycerides or mixtures thereof, lactose, dextrose, sucrose, mannitol, sorbitol, cellulose and/or glycine:

b) a lubricant, e.g., silica, talcum, stearic acid its magnesium or calcium salt and/or polyethylene glycol; for tablets also;

c) a binder, e.g., magnesium aluminum silicate, starch paste, gelatin, tragacanth, methylcellulose, sodium carboxymethylcellulose, natural and synthetic gums such as acacia tragacanth or sodium alginate, waxes and/or polyvinylpyrrolidone, if desired;

d) a disintegrant, e.g., starches, agar, algic acid or its sodium salt, and/or effervescent mixtures;

e) absorbent, colorant, flavorant and/or sweetener;

f) an emulsifier or dispersing agent, e.g. Labrasol, HPMC, labrafil, peceol, capmul, vitamin E TGPS and/or other acceptable emulsifier; and/or

g) an agent that enhances absorption of the compound such as cyclodextrin, hydroxypropyl-cyclodextrin, PEG400, and/or PEG200.

Liquid, particularly injectable, compositions can, for example, be prepared by dissolution, dispersion, etc. For example, a compound of the present invention is dissolved in or mixed with a pharmaceutically acceptable solvent such as, for example, water, saline, aqueous dextrose, glycerol, ethanol, and/or the like, to thereby form an injectable isotonic solution or suspension. Said composition may be sterilized and/or contain adjuvants, such as preserving, stabilizing wetting or emulsifying agents, solution promoters, salts for regulating osmotic pressure and/or buffers.

A compound of the present invention may also be formulated as a suppository that can be prepared from fatty emulsions or suspensions; using polyalkylene glycols such as propylene glycol, as the carrier.

A compound of the present invention may also be delivered by the use of monoclonal antibodies as individual carriers to which the compound is coupled. A compound of the present invention may be coupled with a soluble polymer as a targetable drug carrier. Such polymers can include, but are not limited to, polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, a compound of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphiphillic block copolymers of hydrogels. In one embodiment disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection. In addition, they may also contain other therapeutically valuable substances. Said compositions may be prepared according to conventional mixing, granulating and/or coating methods, respectively, and contain about 0.1-75%, or contain about 1-50%, of the active ingredient.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc, zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing a compound of the present invention in the proper medium. Absorption enhancers can also be used to increase the flux of the compound across the skin. The rate can be controlled by either providing a rate controlling membrane or by dispersing the compound in a polymer matrix or gel.

Compositions of the present invention can be prepared according to conventional mixing, granulating and/or coating methods, respectively, and the present pharmaceutical compositions can contain from about 0.1% to about 99% of compound by weight or volume.

The present invention further provides pharmaceutical compositions and dosage forms that comprise one or more agents that reduce the rate by which a compound of the present invention as an active ingredient will decompose. Such agents, which are referred to herein as “stabilizers” include, but are not limited to, antioxidants such as ascorbic acid, pH buffers, and/or salt buffers, etc.

The dosage regimen utilizing a compound of the present invention may be selected in accordance with a variety of factors including type, species, age, weight, sex and/or medical condition of the subject; the severity of the condition to be treated; the route of administration; the renal or hepatic function of the subject; and the particular disclosed compound employed. A physician, clinician or veterinarian of ordinary skill can readily determine the effective amount of each of the active ingredients necessary to prevent, treat or inhibit the progress of the disorder or disease.

Effective dosage amounts of a compound of the present invention, when used for the indicated effects, range from about 0.5 mg to about 5000 mg of the compound as needed to treat the condition.

In some embodiments, a method of the present invention comprises administering to a subject a compound of the present invention in an amount of about 0.05 to about 5 mg of the compound per kg of the subject, such as, for example, about 0.1 mg/kg to about 5 mg/kg, about 0.1 mg/kg to about 3 mg/kg, about 0.3 mg/kg to about 1 mg/kg, or about 1 mg/kg to about 3 mg/kg. In some embodiments, a compound of the present invention may be administered to a subject in an amount of about 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.25, 1.5, 1.75, 2, 2.25, 2.5, 2.75, 3, 3.25, 3.5, 3.75, 4, 4.25, 4.5, 4.75, or 5 mg of the compound per kg of the subject.

A compound of the present invention may be administered to a subject one or more times per day and/or week (e.g., 1, 2, 3, 4, 5, or more times per day and/or week) for a period of time (e.g., about 1 to about 52 weeks or until a desired therapeutic effect and/or treatment and/or prevention is achieved). In some embodiments, a compound of the present invention is administered to a subject one, two or three times per day. In some embodiments, a compound of the present invention is administered to a subject two or three times a week or every two or three days. In some embodiments, a compound of the present invention is administered to a subject once a day for about 1 to about 52 weeks or until a desired therapeutic effect and/or treatment and/or prevention is achieved.

A compound of the present invention may be administered either simultaneously with, or before or after, one or more other therapeutic agent(s). A compound of the present invention may be administered separately, by the same or different route of administration, or together in the same pharmaceutical composition as the other therapeutic agent(s).

In some embodiments, the invention provides a product comprising a compound of Formula I-II and at least one other therapeutic agent as a combined preparation for simultaneous, separate or sequential use in therapy. In some embodiments, the one or more additional therapeutic agent(s) are an ACE inhibitor, acetyl CoA carboxylase inhibitor, adenosine A3 receptor agonist, adiponectin receptor agonist, AKT protein kinase inhibitor, AMP-activated protein kinases (AMPK), amylin receptor agonist, angiotensin II AT-1 receptor antagonist, autotaxin inhibitors, bioactive lipid, calcitonin agonist, caspase inhibitor, caspase-3 stimulator, cathepsin inhibitor, caveolin 1 inhibitor, CCR2 chemokine antagonist, CCR3 chemokine antagonist, CCR5 chemokine antagonist, chloride channel stimulator, CNR1 inhibitor, cyclin D1 inhibitor, cytochrome P450 7A1 inhibitor, DGAT1/2 inhibitor, dipeptidyl peptidase IV inhibitor, endosialin modulator, eotaxin ligand inhibitor, extracellular matrix protein modulator, farnesoid X receptor agonist, fatty acid synthase inhibitors, FGF1 receptor agonist, fibroblast growth factor (FGF-15, FGF-19, FGF-21) ligands, galectin-3 inhibitor, glucagon receptor agonist, glucagon-like peptide 1 agonist, G-protein coupled bile acid receptor 1 agonist, hedgehog (Hh) modulator, hepatitis C virus NS3 protease inhibitor, hepatocyte nuclear factor 4 alpha modulator (HNF4A), hepatocyte growth factor modulator, HMG CoA reductase inhibitor, IL-10 agonist, IL-17 antagonist, ileal sodium bile acid cotransporter inhibitor, insulin sensitizer, integrin modulator, intereukin-1 receptor-associated kinase 4 (IRAK4) inhibitor, Jak2 tyrosine kinase inhibitor, klotho beta stimulator, 5-lipoxygenase inhibitor, lipoprotein lipase inhibitor, liver X receptor, LPL gene stimulator, lysophosphatidate-1 receptor antagonist, lysyl oxidase homolog 2 inhibitor, matrix metalloproteinases (MMPs) inhibitor, MEKK-5 protein kinase inhibitor, membrane copper amine oxidase (VAP-1) inhibitor, methionine aminopeptidase-2 inhibitor, methyl CpG binding protein 2 modulator, microRNA-21 (miR-21) inhibitor, mitochondrial uncoupler, myelin basic protein stimulator, NACHT LRR PYD domain protein 3 (NLRP3) inhibitor, NAD-dependent deacetylase sirtuin stimulator, NADPH oxidase inhibitor (NOX), nicotinic acid receptor 1 agonist, P2Y13 purinoceptor stimulator, PDE 3 inhibitor, PDE 4 inhibitor, PDE 5 inhibitor, PDGF receptor beta modulator, phospholipase C inhibitor, PPAR alpha agonist, PPAR delta agonist, PPAR gamma agonist, PPAR gamma modulator, protease-activated receptor-2 antagonist, protein kinase modulator, Rho associated protein kinase inhibitor, sodium glucose transporter-2 inhibitor, SREBP transcription factor inhibitor, STAT-1 inhibitor, stearoyl CoA desaturase-1 inhibitor, suppressor of cytokine signalling-1 stimulator, suppressor of cytokine signalling-3 stimulator, transforming growth factor 3 (TGF-β3), transforming growth factor β activated Kinase 1 (TAKi), thyroid hormone receptor beta agonist, TLR-4 antagonist, transglutaminase inhibitor, tyrosine kinase receptor modulator, GPCR modulator, nuclear hormone receptor modulator, WNT modulators, and/or YAP/TAZ modulator.

In some embodiments, the therapy is the treatment or prevention of a disease or condition mediated by a FXR. Products provided as a combined preparation include, but are not limited to, a composition comprising a compound of Formula I-II and one or more therapeutic agent(s) together in the same pharmaceutical composition, or the compound of Formula I-II and one or more therapeutic agent(s) in a separate form, e.g. in the form of a kit.

In some embodiments, a compound of the present invention is an isotopically labelled compound. An “isotopically labelled compound” as used herein refers to a compound in which at least one atomic position is enriched in a specific isotope of the designated element to a level which is significantly greater than the natural abundance of that isotope. For example, one or more hydrogen atom positions in a compound can be enriched with deuterium to a level that is significantly greater than the natural abundance of deuterium, for example, enrichment to a level of at least 1%, preferably at least 20% or at least 50%. Such a deuterated compound may, for example, be metabolized more slowly than its non-deuterated analogue, and therefore exhibit a longer half-life when administered to a subject (Annual Reports In Medicinal Chemistry, Vol. 26, 2011, Chapter 24—Deuterium in Drug Discovery and Development, pages 403-417). Such compounds can be synthesized using methods known in the art, for example, by employing deuterated starting materials. Unless stated to the contrary, isotopically labelled compounds are pharmaceutically acceptable.

The present invention is explained in greater detail in the following non-limiting examples.

### EXAMPLES

The reaction schemes described below are intended to provide a general description of the methodology employed in the preparation of the compounds of the present invention. The examples provided herein are offered to illustrate but not limit the compounds of the present invention, as well as the preparation of such compounds and intermediates

All starting materials, building blocks, reagents, acids, bases, dehydrating agents, solvents and catalysts utilized to synthesize the compounds of the present invention are either commercially available or can be routinely prepared by procedures described in the literature, for example, Houben-Weyl “Science of Synthesis” volumes 1-48, Georg Thieme Verlag, and subsequent versions thereof.

A reaction may be carried out in the presence of a suitable solvent or diluent or of mixture thereof in a manner known to those skilled in the art of organic synthesis. A reaction may also be carried out, if needed, in the presence of an acid or a base, with cooling or heating, for example in a temperature range from about −30° C. to about 150° C. In some embodiments, a reaction is carried out in a temperature range from about 0° C. to about 100° C., and more particularly, in a temperature range from room temperature to about 80° C., in an open or closed reaction vessel and/or in the atmosphere of an inert gas, for example nitrogen.

### Abbreviations

aq. Aqueous
AMP adenosine monophosphate
ATP adenosine triphosphate
Boc tertiary butyl carboxy
cDNA complementary deoxyribonucleic acid
CO₂ carbon dioxide
Cu(I)I copper (I) iodide
d doublet
dd doublet of doublets
DMF dimethylfomamide
DMSO dimethylsulfoxide
Et₃N triethylamine
EtOH ethanol
g gramme
h hour(s)
LCMS liquid chromatography and mass spectrometry
m multiplet
M molar
MeOH methanol
MS mass spectrometry
N Normal
m multiplet
mg milligram
min(s) minute(s)
ml milliliter
m mol
mmol millimol
Na₂SO₄ sodium sulfate
NMR nuclear magnetic resonance
O₂ oxygen
s singlet
tert tertiary
THE tetrahydrofuran
t triplet

### Example 1

### Preparation of 4-((8-azabicyclo[3.2.1]octan-3-yloxy)methyl)-5-cyclopropyl-3-(2-(trifluoromethoxy)phenyl)isoxazole

### (E)-2-(trifluoromethoxy) benzaldehyde oxime

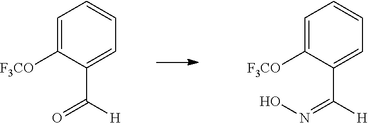 [SMILES: O=Cc1ccccc1OC(F)(F)F ; O/N=C\c1ccccc1OC(F)(F)F]

A solution of sodium hydroxide (3.75 g, 0.093 mol) in water (64 ml) was added to a stirred solution of hydroxylamine hydrochloride (6.3 g, 0.0907 mol) in water (64 ml) at 0° C. After 10 mins, a solution of 2-(trifluoromethoxy) benzaldehyde (15 g, 0.078 mol) in ethanol (64 ml) was added. The resulting solution was allowed to stir for an additional 1 h at room temperature. The resulting solution was diluted with ice water, extracted with ethyl acetate and the combined organic layer was washed with brine, dried over anhydrous sodium sulfate, filtered and the filtrate concentrated under reduced pressure to afford the titled compound (16.5 g, 86%) as a solid. ¹H NMR (400 MHz, d⁶-DMSO): δ 11.75 (s, 1H), 8.22 (s, 1H), 7.60-7.38 (m, 3H). 8.23 (S, 1H), 7.88 (dd, J=8.0 Hz, J=2 Hz, 1H), 7.59-7.51 (m, 1H), 7.49-7.42 (m, 2H).

### (Z)—N-hydroxy-2-(trifluoromethoxy) benzimidoyl chloride

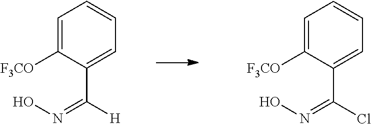 [SMILES: O/N=C\c1ccccc1OC(F)(F)F ; O/N=C(/Cl)c1ccccc1OC(F)(F)F]

N-chlorosuccinimide (12 g, 0.0901 mol) was slowly added to a solution of (E)-2-(trifluoromethoxy) benzaldehyde oxime (16.5 g, 0.0804 mol) in N, N-dimethylformamide (165 ml) at room temperature. After 1 h the solution was diluted with water and extracted with ethyl acetate. The combined organic layers were washed with brine, dried over anhydrous Na₂SO₄, filtered and concentrated under reduced pressure to afford the titled compound (20 g, 92%) as a solid. ¹H NMR (400 MHz, d⁶-DMSO): δ 12.65 (s, 1H), 7.72-7.64 (m, 2H), 7.51-7.49 (m, 2H).

### Methyl 5-cyclopropyl-3-(2-(trifluoromethoxy) phenyl) isoxazole-4-carboxylate

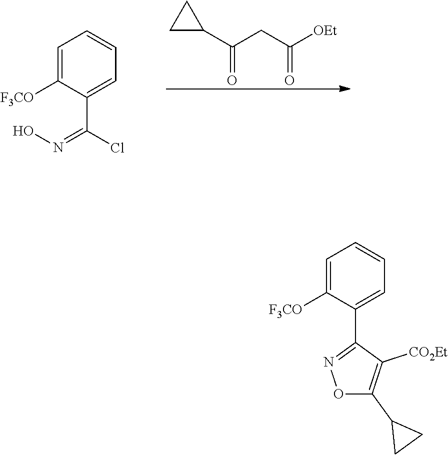 [SMILES: O/N=C(/Cl)c1ccccc1OC(F)(F)F ; CCOC(=O)CC(=O)C1CC1 ; CCOC(=O)c1c(-c2ccccc2OC(F)(F)F)noc1C1CC1]

A solution of methyl 3-cyclopropyl-3-oxopropanoate (26 g, 0.166 mol) in dichloromethane (50 ml) was added to a solution of (Z)-2-(trifluoromethoxy)benzoyl chloride oxime (20 g, 0.083 mol) and triethylamine (100 ml) in dichloromethane (150 ml) at 0° C. After 10 mins the mixture was allowed to warm room temperature and stirred for a further 16 h. The reaction mixture was then diluted with water and dichloromethane, separated and the organic layer dried over anhydrous Na₂SO₄, filtered concentrated under reduced pressure. The crude product was purified by flash column chromatography using silica gel 100-200 mesh eluting with 20% ethyl acetate in petroleum ether to afford the titled compound (12 g, 44%) as a solid. LC-MS: 2.34 mins, [M+H]⁺ 342

### (5-cyclopropyl-3-(2-(trifluoromethoxy) phenyl) isoxazol-4-yl) methanol

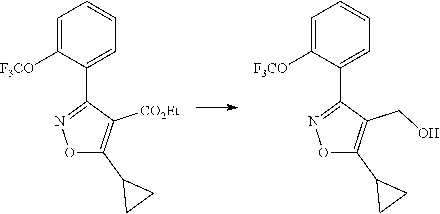 [SMILES: CCOC(=O)c1c(-c2ccccc2OC(F)(F)F)noc1C1CC1 ; OCc1c(-c2ccccc2OC(F)(F)F)noc1C1CC1]

2M Lithium aluminium hydride in THE (50 ml, 0.1009 mol) was added dropwise to a solution of methyl 5-cyclopropyl-3-(2-(trifluoromethoxy) phenyl) isoxazole-4-carboxylate (12 g, 0.035 mol) in tetrahydrofuran (120 ml), under nitrogen at −10° C. After 30 mins ethyl acetate, water and 15% aq. sodium hydroxide were added and the resulting mixture filtered and washed with ethyl acetate. The filtrate was washed with brine, dried over anhydrous Na₂SO₄, filtered and concentrated under reduced pressure to afford the titled compound (9.5 g, 31%) as an oil. LC-MS: 2.03 mins, [M+H]⁺ 300

### 4-(bromomethyl)-5-cyclopropyl-3-(2-(trifluoromethoxy) phenyl) isoxazole

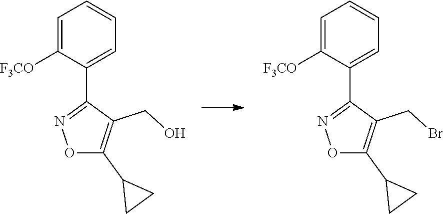 [SMILES: OCc1c(-c2ccccc2OC(F)(F)F)noc1C1CC1 ; FC(F)(F)Oc1ccccc1-c1noc(C2CC2)c1CBr]

A solution of carbon tetrabromide (15.8 g, 0.0476 mol) in dichloromethane (50 ml) was added drop wise to a solution of triphenylphosphine (12.5 g, 0.047 mol) and (5-cyclopropyl-3-(2-(trifluoromethoxy) phenyl) isoxazol-4-yl) methanol (9.5 g, 0.0317 mol) in dichloromethane (100 ml) at room temperature. After 1 h the reaction mixture was concentrated under reduced pressure and purified by flash column chromatography using silica gel 100-200 mesh, eluting with 35% ethyl acetate in petroleum ether to afford the titled compound (7 g, 60%) as an oil. ¹H NMR (400 MHz, CDCl₃): δ 7.64-7.53 (m, 2H), 7.46-7.38 (m, 2H), 4.33 (s, 2H), 2.16-2.06 (m, 1H), 1.32-1.23 (m, 2H), 1.22-1.15 (m, 2H).

### 4-((8-azabicyclo[3.2.1]octan-3-yloxy)methyl)-5-cyclopropyl-3-(2-(trifluoromethoxy)phenyl)isoxazole

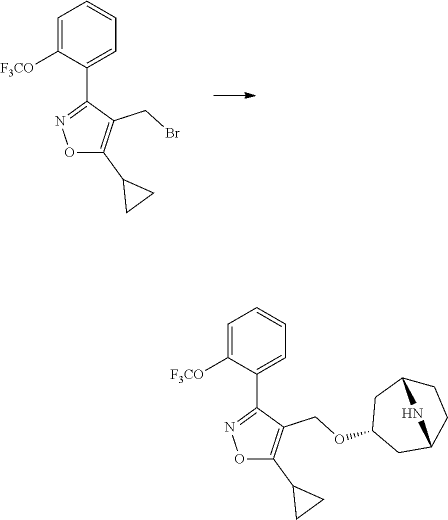 [SMILES: FC(F)(F)Oc1ccccc1-c1noc(C2CC2)c1CBr ; FC(F)(F)Oc1ccccc1-c1noc(C2CC2)c1CO[C@H]1C[C@H]2CC[C@@H](C1)N2]

18-Crown-6 (5.6 g, 0.021 mol) and potassium tert-butoxide (4.7 g, 0.042 mol) were added to a solution of N-Boc-nortropine (4.8 g, 0.021 mol) in tetrahydrofuran (100 ml). After 1 h, a solution of 4-(bromomethyl)-5-cyclopropyl-3-(2-(trifluoromethoxy)-phenyl) isoxazole (7 g, 0.019 mol) in tetrahydrofuran (40 ml) was added drop wise at room temperature. After 16 h the reaction mixture was concentrated, diluted with water and ethyl acetate, separated and the organic layer washed with brine, dried over anhydrous Na₂SO₄, filtered and concentrated under reduced pressure. The crude product was purified by flash column chromatography using silica gel 100-200 mesh eluting with 0-100% ethyl acetate in petroleum ether to afford tert-butyl 3-((5-cyclopropyl-3-(2-(trifluoromethoxy)phenyl)isoxazol-4-yl)methoxy)-8-azabicyclo[3.2.1]octane-8-carboxylate (7 g, 71%) as an oil. This was dissolved in dichloromethane (80 ml) and trifluoroacetic acid (20 ml) was added at room temperature. After 1 h, the reaction mixture was evaporated under reduced pressure, the residue was dissolved in ethyl acetate, washed with a saturated sodium bicarbonate solution, the organic layer was then dried over anhydrous Na₂SO₄, filtered and the filtrate was concentrated under reduced pressure to afford the titled compound (5.4 g, 70%) as a colourless oil. LC-MS: 1.81 mins, [M+H]⁺ 409

### Example 2

### Preparation of Sodium 2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-sulfinate 2,6-dibromo-4-fluorobenzo[d]thiazole

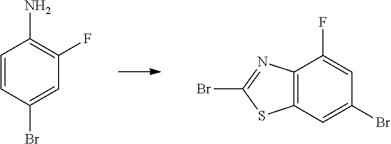 [SMILES: Nc1ccc(Br)cc1F ; Fc1cc(Br)cc2sc(Br)nc12]

A solution of bromine (7 ml, 0.131 mol) in acetic acid (50 ml) was added to a solution of 4-bromo-2-fluoro aniline (25 g, 0.131 mol) and sodium thiocyanide (43 g, 0.526 mol) in acetic acid (200 ml) at 0° C. and the resulting mixture then warmed to 40° C. After 16 h the reaction mixture was diluted with ice water and the pH adjusted to 8-9 with ammonium hydroxide solution. The resulting mixture was filtered and the remaining solid dried to afford crude 6-bromo-4-fluorobenzo[d]thiazol-2-amine as a solid. The product was dissolved in acetonitrile (60 ml) at 0° C. before tert-butyl nitrite (3.6 ml) and then copper (II) bromide (5.41 g, 0.024 mol) were added. The reaction mixture was warmed to 40° C. and after 16 h the reaction mixture was diluted with ethyl acetate, washed with water and the organic layer dried over anhydrous Na₂SO₄, filtered and concentrated under reduced pressure to afford the titled compound (4 g, 10%) as a solid. LC-MS: 2.37 mins, [M+H]⁺ 310

### 4-((8-(6-bromo-4-fluorobenzo[d]thiazol-2-yl)-8-azabicyclo[3.2.1]octan-3-yloxy)methyl)-5-cyclopropyl-3-(2-(trifluoromethoxy)phenyl)isoxazole

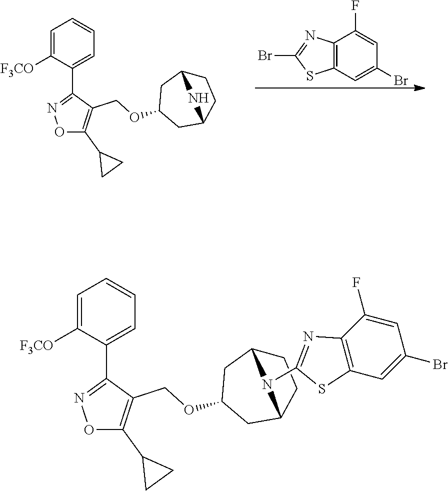 [SMILES: FC(F)(F)Oc1ccccc1-c1noc(C2CC2)c1CO[C@H]1C[C@H]2CC[C@@H](C1)N2 ; Fc1cc(Br)cc2sc(Br)nc12 ; Fc1cc(Br)cc2sc(N3[C@@H]4CC[C@H]3C[C@@H](OCc3c(-c5ccccc5OC(F)(F)F)noc3C3CC3)C4)nc12]

Potassium carbonate (0.86 g, 0.0062 mol) was added to a solution of 4-((8-azabicyclo[3.2.1]octan-3-yloxy)methyl)-5-cyclopropyl-3-(2-(trifluoromethoxy)phenyl) isoxazole (0.85 g, 0.0020 mol) and 2,6-dibromo-4-fluorobenzo[d]thiazole (0.64 g, 0.0020 mol) in DMF (16 ml) at room temperature. After 16 h, the reaction mixture was poured into ice water and extracted with ethyl acetate. The combined organic layers were washed with brine, dried over anhydrous Na₂SO₄, filtered, concentrated under reduced pressure and purified by column chromatography using silica gel 100-200 mesh eluting with 80% ethyl acetate in petroleum ether to afford 4 the titled compound (0.75 g, 57%) as a solid. ¹H NMR (400 MHz, d⁶-DMSO): δ 7.88-7.85 (m, 1H), 7.72-7.62 (m, 2H), 7.58-7.54 (m, 2H), 7.41 (dd, J=10.4 Hz, J=2 Hz, 1H), 4.34 (s, 2H), 4.22-4.15 (m, 2H), 3.59-3.51 (m, 1H), 2.40-2.28 (m, 1H), 2.02-1.92 (m, 2H), 1.86-1.78 (m, 4H), 1.77-1.68 (m, 2H), 1.18-1.04 (m, 4H).

### Methyl 3-(2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazol-6-ylsulfonyl)propanoate

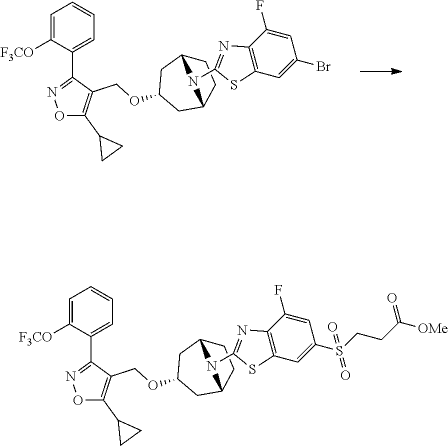 [SMILES: Fc1cc(Br)cc2sc(N3[C@@H]4CC[C@H]3C[C@@H](OCc3c(-c5ccccc5OC(F)(F)F)noc3C3CC3)C4)nc12 ; COC(=O)CCS(=O)(=O)c1cc(F)c2nc(N3[C@@H]4CC[C@H]3C[C@@H](OCc3c(-c5ccccc5OC(F)(F)F)noc3C3CC3)C4)sc2c1]

Copper(I) iodide (0.19 g, 0.0010 mol) was added to a solution of 4-((8-(6-bromo-4-fluorobenzo[d]thiazol-2-yl)-8-azabicyclo[3.2.1]octan-3-yloxy)methyl)-5-cyclopropyl-3-(2-(trifluoromethoxy)phenyl)isoxazole (0.65 g, 0.0010 mol), sodium 3-methoxy-3-oxopropane-1-sulfinate (0.35 g, 0.0020 mol) and L-proline (0.117 g, 0.0010 mol) in dimethylsulfoxide (13 ml) at room temperature and the resulting mixture warmed to 130° C. After 16 h, water and ethyl acetate were added and the mixture filtered through celite. The filtrate was then washed with cold water, brine, dried over anhydrous Na₂SO₄, filtered, concentrated under reduced pressure and purified by flash column chromatography using silica gel 100-200 mesh, eluting with 0-70% ethyl acetate in petroleum ether to afford the titled compound (0.15 g, 20%) as a solid. LC-MS: 2.50 mins, [M+H]⁺ 710

### Sodium 2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-sulfinate

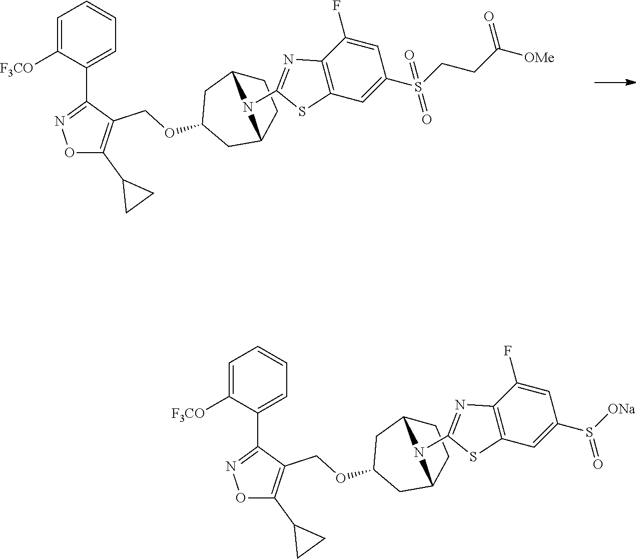

1M Sodium methoxide in methanol (0.5 ml) was added to a solution of methyl 3-(2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazol-6-ylsulfonyl) propanoate (0.15 g, 0.0002 mol) in methanol (2 ml) at 0° C. After 5 h at room temperature, the reaction mixture was concentrated, triturated with diethyl ether, washed with n-pentane and dried under reduced pressure to afford the titled compound (0.102 g, 75%) as a solid. LC-MS: 4.85 mins, [M-Na+H]⁺ 624; ¹H NMR (400 MHz, d⁶-DMSO): δ 7.72-7.62 (m, 2H), 7.59-7.52 (m 3H), 7.09 (d, J=10.4 Hz, 1H), 4.33 (s, 2H), 4.18-4.12 (m, 2H), 3.58-3.54 (m, 1H), 2.40-2.30 (m, 1H), 2.06-1.96 (m, 2H), 1.88-1.76 (m, 4H), 1.74-1.66 (m, 2H), 1.18-1.06 (m, 4H).

### Example 3

### 2,6-Dibromo-4-methylbenzo[d]thiazole

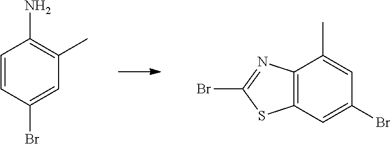

A solution of bromine (2.7 ml, 53.76 mmol) in acetic acid (20 ml) was added to a solution of 4-bromo-2-fluoro aniline (10 g, 53.76 mmol) and sodium thiocyanide (17.4 g, 215 mmol) in acetic acid (80 ml) at 0° C. and the resulting mixture then warmed to 40° C. After 16 h the reaction mixture was diluted with ice water and the pH adjusted to 8-9 with ammonium hydroxide solution. The resulting mixture was filtered and the remaining solid dried to afford crude 6-bromo-4-methylbenzo[d]thiazol-2-amine (3.2 g) as a solid. The 6-bromo-4-methylbenzo[d]thiazol-2-amine (3 g) was dissolved in acetonitrile (60 ml) at 0° C. before tert-butyl nitrite (3.84 ml) and then a solution of copper (II) bromide (5.88 g, 223 mmol) in acetonitrile (60 ml) were added. The reaction mixture was warmed to 40° C. and after 16 h the reaction mixture was diluted with ethyl acetate, washed with water and the organic layer dried over anhydrous Na₂SO₄, filtered and concentrated under reduced pressure to afford the titled compound (1.4 g, 35%) as a solid. LC-MS: 2.49 mins, [M+H]⁺ 308

### 4-((8-(6-bromo-4-methylbenzo[d]thiazol-2-yl)-8-azabicyclo[3.2.1]octan-3-yloxy)methyl)-5-cyclopropyl-3-(2-(trifluoromethoxy)phenyl)isoxazole

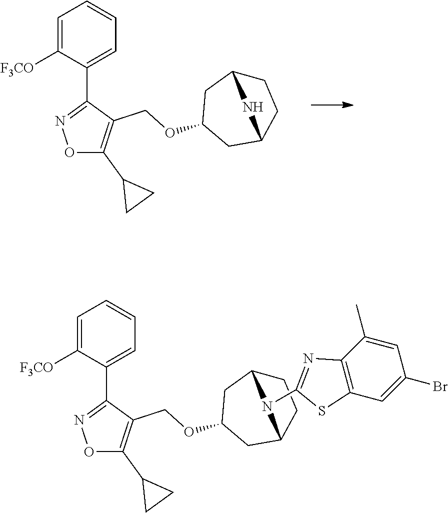 [SMILES: FC(F)(F)Oc1ccccc1-c1noc(C2CC2)c1CO[C@H]1C[C@H]2CC[C@@H](C1)N2 ; Cc1cc(Br)cc2sc(N3[C@@H]4CC[C@H]3C[C@@H](OCc3c(-c5ccccc5OC(F)(F)F)noc3C3CC3)C4)nc12]

Potassium carbonate (1.5 g, 11.02 mmol) was added to a stirred solution of 4-((8-azabicyclo[3.2.1]octan-3-yloxy)methyl)-5-cyclopropyl-3-(2-(trifluoromethoxy)phenyl)isoxazole (1.5 g, 3.67 mmol) and 2,6-Dibromo-4-methylbenzo[d]thiazole (1.12 g, 3.67 mmol) in N,N-dimethylformamide (20 ml) at room temperature. After 16 h the reaction mixture was poured into ice water, extracted with ethyl acetate, the combined organic layers washed with brine, dried over anhydrous Na₂SO₄, filtered, concentrated under reduced pressure and purified by flash column chromatography using silica gel 100-200 mesh, eluting with 73% ethyl acetate in petroleum ether to afford the titled compound (1.06 g, 46%) as a solid. LC-MS: 3.06 mins, [M+H]⁺ 636

### Methyl 3-(2-(3-((5-cyclopropyl-3-(2-(trifluoromethoxy)phenyl)isoxazol-4-yl) methoxy)-8-azabicyclo[3.2.1]octan-8-yl)-4-methylbenzo[d]thiazol-6-ylsulfonyl)propanoate

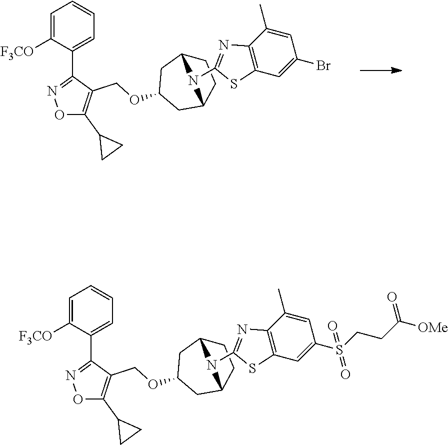 [SMILES: Cc1cc(Br)cc2sc(N3[C@@H]4CC[C@H]3C[C@@H](OCc3c(-c5ccccc5OC(F)(F)F)noc3C3CC3)C4)nc12 ; COC(=O)CCS(=O)(=O)c1cc(C)c2nc(N3[C@@H]4CC[C@H]3C[C@@H](OCc3c(-c5ccccc5OC(F)(F)F)noc3C3CC3)C4)sc2c1]

Cu(I)I (570 mg, 3.02 mmol) was added to a solution of 4-((8-(6-bromo-4-methylbenzo[d]thiazol-2-yl)-8-azabicyclo[3.2.1]octan-3-yloxy)methyl)-5-cyclopropyl-3-(2-(trifluoromethoxy)phenyl)isoxazole_(960 mg, 1.51 mmol), sodium 3-methoxy-3-oxopropane-1-sulfinate (790 mg, 4.54 mmol) and L-proline (174 mg, 1.51 mmol) in dimethylsulfoxide (20 ml) at room temperature and the resulting mixture warmed to 130° C. After 16 h, water and ethyl acetate were added and the mixture filtered through celite. The filtrate was then washed with cold water, brine, dried over anhydrous Na₂SO₄, filtered, concentrated under reduced pressure and purified by flash column chromatography using silica gel 100-200 mesh, eluting with 0-65% ethyl acetate in petroleum ether to afford the titled compound (0.25 g, 25%) as a solid. LC-MS: 2.57 mins, [M+H]⁺ 706

### Sodium 2-(3-((5-cyclopropyl-3-(2-(trifluoromethoxy)phenyl)isoxazol-4-yl)methoxy)-8-azabicyclo[3.2.1]octan-8-yl)-4-methylbenzo[d]thiazole-6-sulfinate

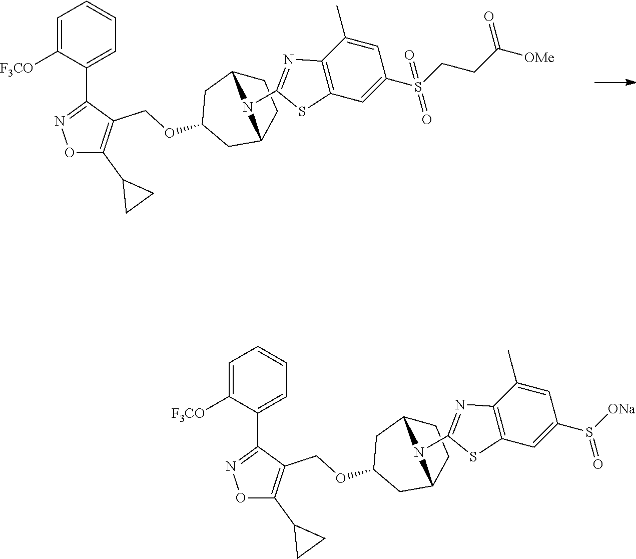

1M Sodium methoxide in methanol (7 ml) was added to a solution of methyl 3-(2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazol-6-ylsulfonyl) propanoate (0.25 g, 0.35 mmol) in methanol (2.5 ml) at 0° C. After 5 h at room temperature, the reaction mixture was concentrated, triturated with diethyl ether, washed with n-pentane and dried under reduced pressure to afford the titled compound (0.112 g, 51%) as a solid. LC-MS: 4.91 mins, [M-Na]+618; ¹H NMR (400 MHz, d⁶-DMSO): 7.72-7.62 (m, 2H), 7.59-7.52 (m, 3H), 7.16-7.12 (m, 1H), 4.33 (s, 2H), 4.20-4.10 (m, 2H), 3.58-3.51 (m, 1H), 2.42 (s, 3H), 2.39-2.30 (m, 1H), 2.08-1.98 (m, 2H), 1.85-1.77 (m, 4H), 1.75-1.65 (m, 2H), 1.18-1.05 (m, 4H).

### Example 4

### Human Farnesoid X Receptor (NR1H4, FXR) Reporter Assay

Determination of a ligand mediated Gal4 promoter driven transactivation to quantify ligand binding mediated activation of FXR. A FXR Reporter Assay kit was purchased from Indigo Bioscience (#IB00601-32) to determine the potency and efficacy of compounds that can induce FXR activation. The nuclear receptor assay system utilizes non-human mammalian cells engineered to provide constitutive high level expression of Human FXR receptor (NR1H4), a ligand-dependent transcription factor. The reporter cells include luciferase reporter gene functionality linked to an FXR-response promoter. Quantifying changes in luciferase expression in the treated reporter cells provides a sensitive surrogate measure of the changes in FXR activity.

The reporter cells incorporate the cDNA encoding beetle luciferase, a protein that catalyses the mono-oxidation of D-luciferin in a Mg²⁺ dependent reaction that consumes O₂ and ATP as co-substrates, yielding the products Oxylucifern, AMP, PPᵢ, CO₂ and photon (light) emission. Luminescence intensity of the reaction is quantified using a luminometer, and is reported in terms of Relative Light Units (RLU's).

The assay was performed according to the manufacturer's instructions. In brief, the test compounds were weighed and dissolved in phosphate buffer solution as a 10 mM stock and diluted to the appropriate concentration using Compound Screening Medium (CSM). The frozen reporter cells were thawed and suspended in Cell Recovery Medium at 37° C. The compounds were then immediately added to the cell plate and the plate was placed into a 37° C., humidified CO₂ incubator for 22-24 h. After incubation, the media was carefully removed, Luciferase detection solution was added and the plate read in a Luminometer. The data was calculated as per kit instructions, with increases in luminescence being directly proportional to increases in compound agonist activity.

Table 1 summarizes the potency ranges for the compounds of the invention. The EC₅₀ values were determined using the Human FXR (NR1H4) assay and efficacy was normalized to GW4064 set as 100%.

TABLE 1
Potency of compounds tested.

| Example | EC50 (μM) | Efficacy (%) |
|---|---|---|
| 2 | 0.46 | 119 |
| 3 | 0.23 | 111 |

### Example 5

### Gene Expression in Human Primary Hepatocytes

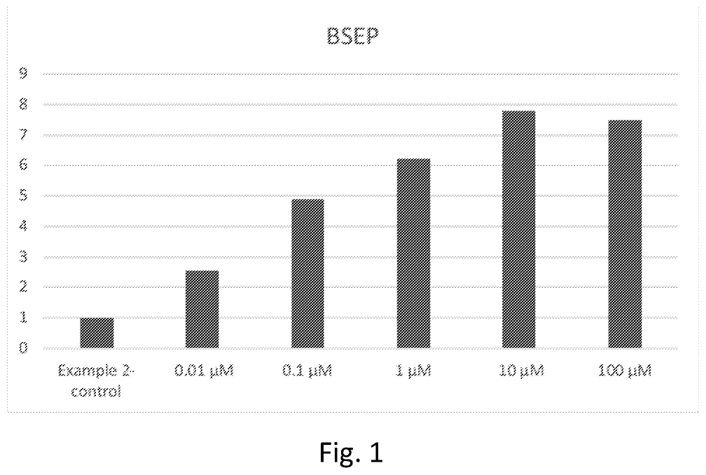

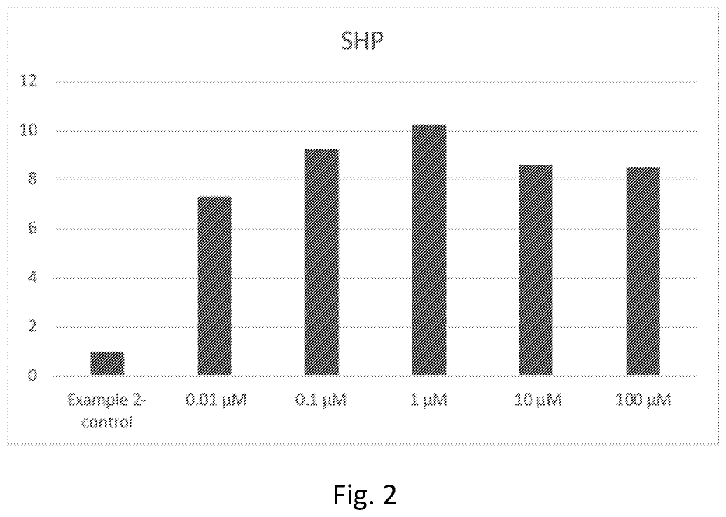

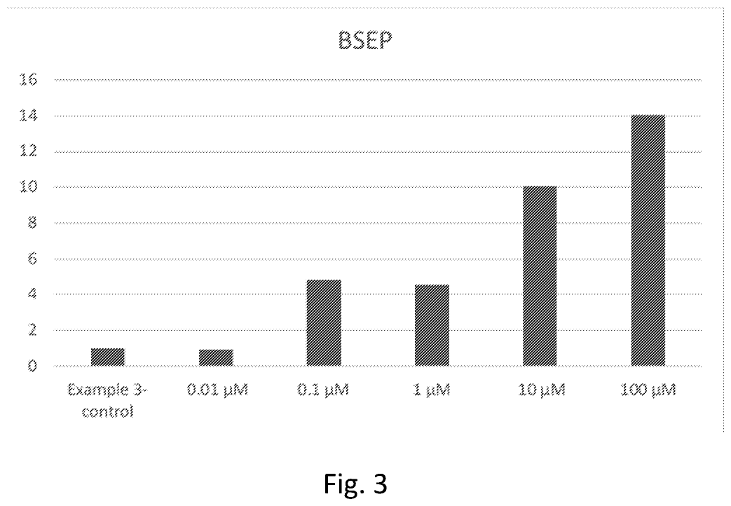

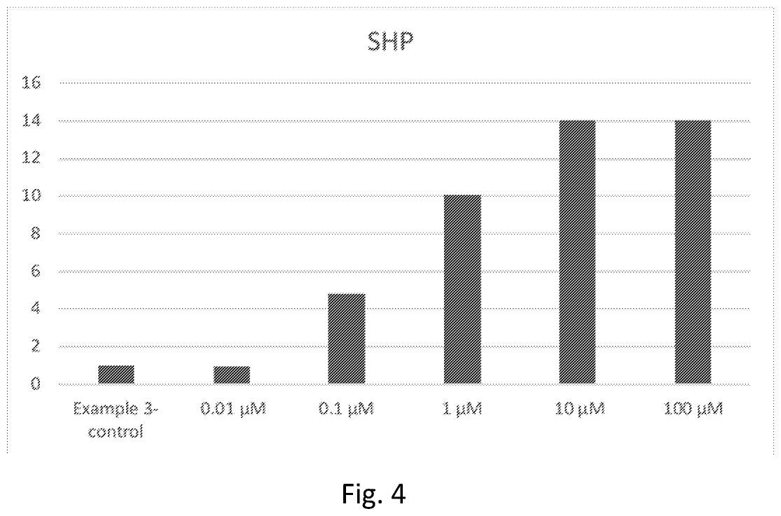

The test compound was dissolved in buffer to give a 10 mM stock solution. The compound was serially diluted to the desired concentrations and then incubated with human primary hepatocytes for 24 hours at 37° C. in a CO₂ incubator. After incubation, the media was removed and the RNA harvested using the Trizol-CHCl₃ method and quantified using Nano drop. The RNA was then Reverse Transcribed to cDNA using a superscript vilo cDNA synthesis kit. The cDNA with respective Taqman primers, master mix are then amplified using quantitative RT-PCR system. FIG. 1 shows the fold change of BSEP gene expression on incubation with the compound of example 2 with primary human hepatocytes. FIG. 2 shows the fold change of SHP gene expression on incubation with the compound of example 2 with primary human hepatocytes. FIG. 3 shows the fold change of BSEP gene expression on incubation with the compound of example 3 with primary human hepatocytes. FIG. 4 shows the fold change of SHP gene expression on incubation with the compound of example 3 with primary human hepatocytes.

The compounds of examples 2 and 3 upregulate FXR related and disease relevant genes on incubation with primary human hepatocytes. BSEP (Bile Salt Excretory Pump) is a direct target for FXR and is strongly induced by FXR agonists. It is a uni-directional, ATP-dependent efflux transporter that plays an important role in the elimination of bile salts from the hepatocyte into the bile canaliculi for export into the gastrointestinal tract. FXR also induces the expression of small heterodimer partner (SHP) which controls a complex set of genes in multiple metabolic pathways. It reduces the expression of CYP7A1, a liver-specific enzyme that catalyzes the first and rate-limiting step in one of the two pathways for bile acid biosynthesis.

### Example 6

### In-Vivo Efficacy in High Fat High Cholesterol Diet (HFHC) Fed Male C57BL/6 Mice

C57BL/6J male mice were purchased at the age of 8 weeks. After an acclimation period of 1 week, animals were maintained on high fat high cholesterol diet (Research diets) with 60% kcal from fat plus 0.5% (w/w) extra cholesterol (Research diet, D02051401) for a period of 4 weeks. Age matched control animals (n=6) were maintained on normal chow. Mice were dosed with vehicle, test compounds and GW4064 morning (7.00 AM) and evening (5:00 PM) from day 0 to 27. At 28^{th} day, animals were dosed at 7:00 AM and kept on fasting for next 4 hours before blood and tissues collection. Plasma samples separated from collected blood were stored at −20° C. immediately for estimation analysis. The samples were analyzed using the Siemens Dimension RxL Max Clinical chemistry analyzer.

Right liver lobe was used for the test compounds exposure, triglyceride and cholesterol estimation. Liver tissues were dissected, weighed and snap frozen and stored at −80° C. for further analysis of hepatic triglyceride, cholesterol and compound exposure. In brief, stored tissues were thawed to room temperature and triglycerides were extracted by homogenizing 25 mg of liver tissues in 1 ml of 5% Triton X100 (Lot No. 61K01741-Sigma Aldrich) in water. Homogenized tissues were slowly heated to 80° C. in water bath for 5 minutes. The samples were cooled down and again heated to solubilize all the triglycerides into solution. The samples were centrifuged for 5 minutes at 4000 RPM and supernatant was collected for quantification³ using commercially available kit from ERBA (Lot No. B061832).

For hepatic cholesterol estimation, ˜10 mg of liver tissue was taken and extracted with ˜200 μL of chloroform (Lot No. SHBG1163V-Sigma Aldrich): isopropanol (Lot No. SHBG8515V-Sigma Aldrich): NP-40 (Lot No. 0001436781-Sigma Aldrich) (7:11:0.1) in a micro homogenizer. Homogenized samples were centrifuge at 13000 g for 10 minutes to remove insoluble material. The organic phase was separated into new tube and air dried at 50° C. to remove chloroform. Thereafter, samples were vacuumed for 30 minutes to remove residue organic solvent. Dried lipids were dissolved in 200 μL of cholesterol assay buffer (provided with Sigma cholesterol assay kit Lot No. B2J210603V), sonicated for 20 minutes and vortexed until mixture was homogenous as mentioned in kit protocol. Samples were diluted with cholesterol assay buffer at (1:10) ratio to fit in standard curve and finally 50 μL of diluted samples were used for the assay.

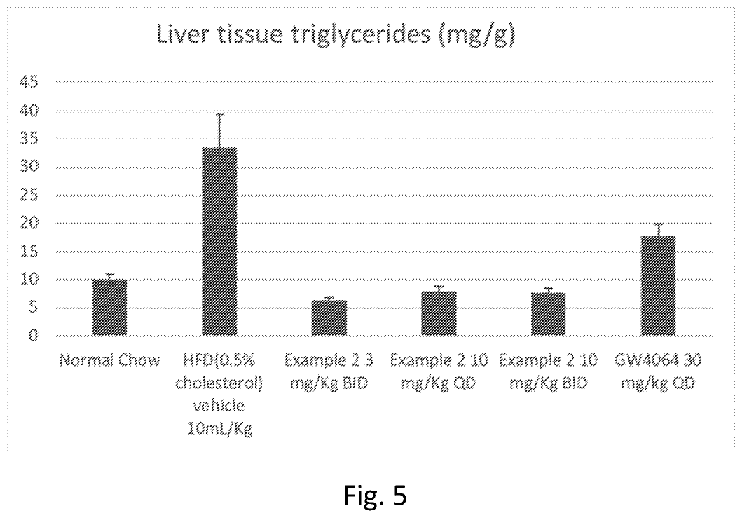

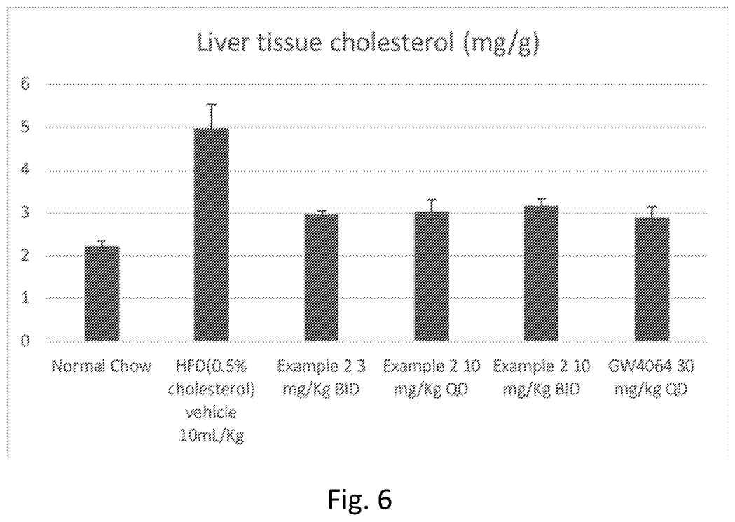

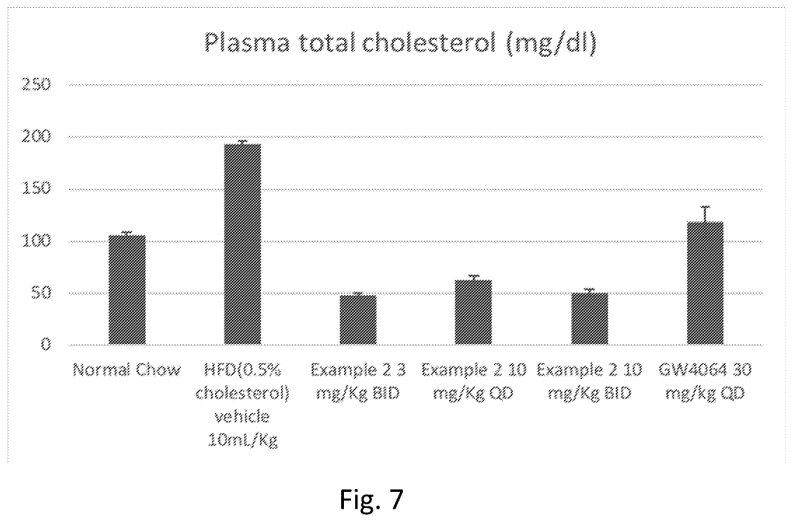

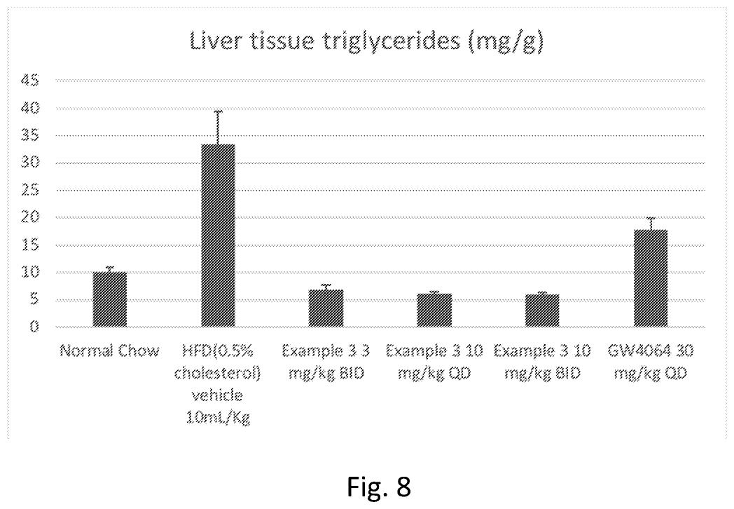

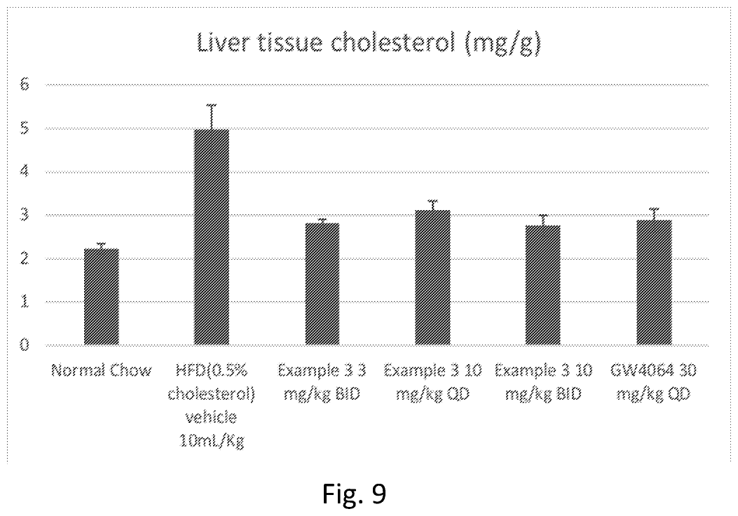

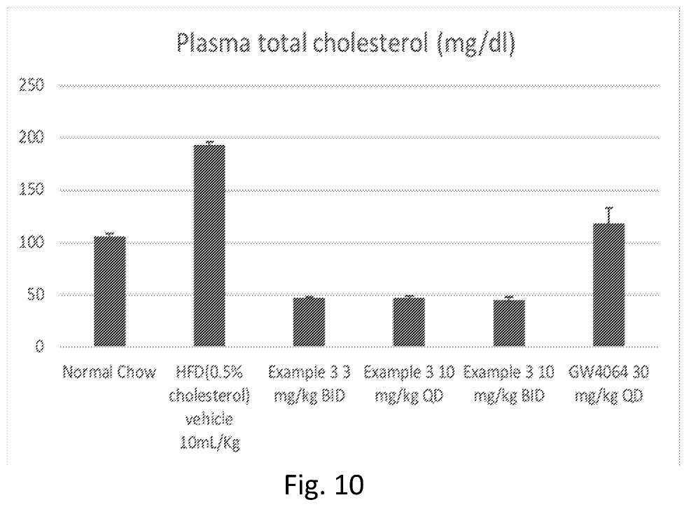

FIG. 5 shows liver triglyceride content in HFHC male mice after 28 days of treatment with the compound of Example 2. FIG. 6 shows liver cholesterol content in HFHC male mice after 28 days of treatment with the compound of Example 2. FIG. 7 shows plasma total cholesterol content in HFHC male mice after 28 days of treatment with the compound of Example 2. FIG. 8 shows liver triglyceride content in HFHC male mice after 28 days of treatment with the compound of Example 3. FIG. 9 shows liver cholesterol content in HFHC male mice after 28 days of treatment with the compound of Example 3. FIG. 10 shows plasma total cholesterol content in HFHC male mice after 28 days of treatment with the compound of Example 3.

The compounds of examples 2 and 3 were orally dosed to HFHC fed male mice at 3 mg/kg BID, 3 mg/kg QD and 10 mg/kg BID. After 28 days reductions in liver triglycerides were observed as well as cholesterol levels in both the liver and plasma. Compound levels were also measured in the plasma and liver to understand its distribution profile. Table 2 summarizes mean compound exposure in the plasma and liver 4 hours after the last dose of the test compound on day 28. The calculated ratio of which show a high exposure in the liver relative to in the plasma. Compounds of the invention may reduce liver triglycerides and/or cholesterol in the liver and/or plasma of a subject. Compounds of the invention may show a distribution profile of high exposure in the liver compared to the plasma.

TABLE 2
Mean compound exposure in plasma and liver

| Example | Dose | Plasma ng/ml | Liver ng/g | Ratio |
|---|---|---|---|---|
| 2 | 3 mg/kg BID | 1.0 | 54.8 | 54.8 |
| 2 | 10 mg/kg QD | 1.2 | 80.1 | 66.8 |
| 2 | 10 mg/kg BID | 2.1 | 138.3 | 65.9 |
| 3 | 3 mg/kg BID | 3.7 | 88.9 | 24.0 |
| 3 | 10 mg/kg QD | 5.9 | 176.3 | 29.9 |
| 3 | 10 mg/kg BID | 11.5 | 198.3 | 17.2 |

### Example 7

### In-Vivo Efficacy in STAM™ Model of Non-Alcoholic Steatohepatitis

NASH was induced in male mice by a single subcutaneous injection of 200 μg streptozotocin (STZ, Sigma-Aldrich, USA) solution 2 days after birth and feeding with high fat diet (HFD, 57 kcal % fat, Cat #HFD32, CLEA Japan, Inc., Japan) after 4 weeks of age. NASH model mice were randomized into groups of 8 mice at 6 weeks of age based on their body weight the day before the start of treatment. The compound of Example 2 was then administered orally in a volume of 10 ml/kg at dose levels 0.3 mg/kg, 1 mg/kg and 3 mg/kg once daily for 3 weeks.

At study termination, two hours fasting blood was collected in polypropylene tubes with anticoagulant (Novo-Heparin, Mochida Pharmaceutical Co. Ltd., Japan) and centrifuged at 1,000×g for 15 minutes at 4° C. The supernatant was collected and stored at −80° C. until use.

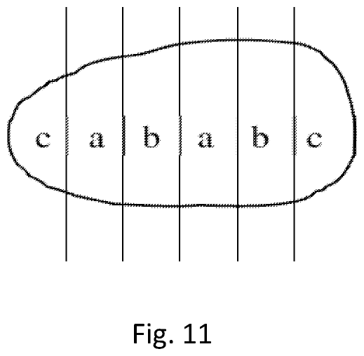

Liver samples were prepared in the following manner—after sacrifice, whole liver was collected and washed with cold saline. The left lateral lobes of livers were separated and dissected as described in FIG. 11 and stored as described below.

For section a, liver specimens were stored at −80° C. embedded in Optimal Cutting Temperature (OCT, Sakura Finetek Japan, Japan) compound for further analysis or shipping.

For section b, liver specimens were fixed in Bouin's solution (Sigma-Aldrich Japan, Japan) for 24 hours. After fixation, these specimens were proceeded to paraffin embedding for HE and Sirius redstaining.

For section c, liver specimens were snap frozen in liquid nitrogen and stored at −80° C. for further analysis or shipping

For HE staining, sections were cut from paraffin blocks of liver tissue prefixed in Bouin's solution and stained with Lillie-Mayer's Hematoxylin (Muto Pure Chemicals Co., Ltd., Japan) and eosin solution (Wako Pure Chemical Industries). NAFLD Activity score (NAS) was calculated according to the criteria of Kleiner (Kleiner D E. et al., Hepatology, 2005; 41:1313). For scoring of NAS, bright field image of HE-stained sections was captured using a digital camera (DFC295; Leica, Germany) at 50- and 200-fold magnifications. Steatosis score in 1 section/mouse (representative 1 field around the central vein at 50-fold magnification), inflammation score in 1 section/mouse (representative 1 field around the central vein at 200-fold magnification) and ballooning score in 1 section/mouse (representative 1 field around the central vein at 200-fold magnification) were estimated.

To visualize collagen deposition, Bouin's fixed liver sections were stained using picro-Sirius red solution (Waldeck, Germany). Briefly, sections were deparaffinized and hydrophilized with xylene, 100-70% alcohol series and RO water, and then treated with 0.03% picro-Sirius red solution (Cat No.: 1A-280) for 60 minutes. After washing through 0.5% acetic acid solution and RO water, stained sections were dehydrated and cleared with 70-100% alcohol series and xylene, then sealed with Entellan® new (Merck, Germany) and used for observation. For quantitative analysis of fibrosis area, bright field images of Sirius red-stained sections were captured around the central vein using a digital camera (DFC295; Leica, Germany) at 200-fold magnification, and the positive areas in 5 fields/section were measured using ImageJ software (National Institute of Health, USA).

The NAFLD activity score (NAS) is a numerical score and is the sum of the separate scores for steatosis (excess hepatic fat storage), hepatocellular ballooning (cell swelling and enlargement) and lobular inflammation (macrophage and lymphocyte aggregation). The score can be used to quantify changes in NAFLD during disease progression and on administration of potential therapeutics. In order to quantify the degree of fibrosis Sirius red stained liver sections were used to visualise and study collagen distribution.

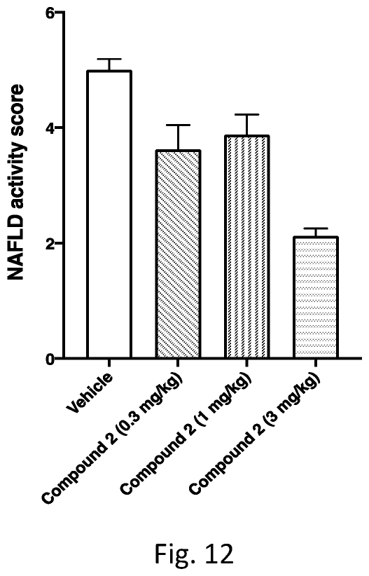

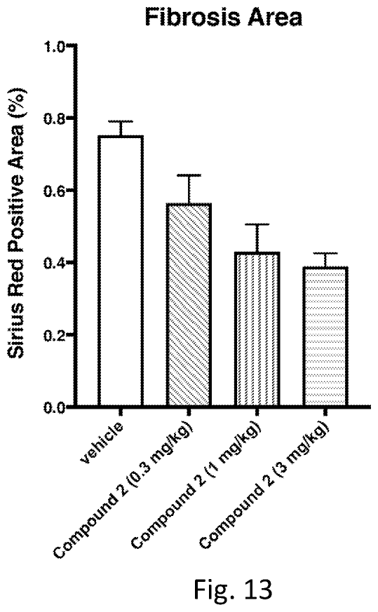

Significant reductions in the NAS score and fibrosis area were observed in the STAM model when the animals were treated with compound 2 once daily for three weeks. FIG. 12 shows the NAFLD Activity score in a STAM model when compound of Example 2 is administered once daily at a 0.3 mg/kg, 1 mg/kg, or 3 mg/kg dose for 3 weeks. FIG. 13 shows the Fibrosis areas in a STAM model when compound of Example 2 is administered once daily at a 0.3 mg/kg, 1 mg/kg, or 3 mg/kg dose for 3 weeks.

### Example 8

### Metabolism in Human Microsomes

Microsomal mixes (247.5 μL) of human microsomes (1250 μL, 4 mg/ml), potassium phosphate buffer (1250 μL), alamethecin (12.5 μL, 5 mg/ml) and cofactors Nicotinamide Adenine Dinucleotide Phosphate NADPH (1250 μL, 4 mM)/uridine 5′-diphospho-glucuronic acid UDPGA (1250 μL, 4 mM) or UDPGA only were loaded onto a shaker for 10 minutes. The test compound (2.5 μL, 100 μM) was then separately added to both microsomal mixes (i.e., one mix with NADPH and one mix without NADPH), incubated at 37° C. and sampled at predetermined time points. Analysis was performed using a AB SCIEX QTRAP 4500 LC-MS/MS, a Kinetex C18, 50*4.6 mm, 5 μm column and eluting with 10 mM Ammonium Acetate/Methanol.

Table 3 summarizes the % of compound remaining after 45 minutes of incubation with human microsomes either in the presence of co-factors UDPGA and NADPH or UDPGA only.

TABLE 3
Percent of compound remaining after
45 minutes of incubation.

| | % remaining | % remaining |
|---|---|---|
| | @ 45 mins of | @ 45 mins of |
| | incubation with | incubation with |
| Compound of Example # | UDPGA and NADPH | UDPGA |
| 2 | 52 | 94 |

Significant metabolism by human microsomes only occurs in the presence of the co-factor NADPH, which shows that the compounds are metabolised via a cyp oxidation pathway. Stability of the compound in the presence of the co-factor UDPGA shows that metabolism via acyl-glucuronidation is not occurring.

### Example 9

### Metabolic Identification in Hepatocytes

A 10 mM stock of test compound from Example 2 (sodium 2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-sulfinate) was prepared in DMSO. 20 μM of final working stock was then prepared by diluting 4 μL of 10 mM stock in 1996 μL of Krebs-Hensleit buffer. 200 μL of hepatocyte cell suspension (2×10⁶ cells/mL) was added to TPP 48 well plate and preincubated for 30 min @ 37° C. in incubator. 200 μL of 20 μM working stock of test compound was added to the cell suspension and incubated in an incubator on vibramax at a shaking speed of 500 rpm. For the 0 min sample, 25 μL of hepatocyte suspension was precipitated with 200 μL of acetonitrile and 25 μL of 20 μM test compound was added. The reaction was stopped at 15, 30, 60, 90, 120 min by precipitating 50 μL of incubation mixture with 200 μL of acetonitrile. The samples were vortexed for 5 min at 1200 rpm and centrifuged at 4000 rpm for 10 min. The supernatant was separated, diluted 2 fold with water and analysed using a HPLC (Shimadzu SIL HTS) and mass spectrometer (5500 Qtrap). Acetonitrile and 0.1% formic acid in Milli-q-water were used as the mobile phase, using a column of either Waters Xbridge C18, 250×3.0 mm, 5.0μ particle size or Phenomnex kinetex 5μ EVO C18 100 A, 50×4.6 mm 5.0μ particle size.

Table 4 summarizes the major metabolites observed when the compound of Example 2 of the invention was incubated in human, dog or mouse hepatocytes.

TABLE 4
Metabolic identification in hepatocytes with the compound of Example 2

| Species | Observed Major Metabolites | Acyl-Glucuronide |
|---|---|---|
| Human | Hydroxy, Alkenyl and Alkenyl-hydroxy | No |
| Dog | Hydroxy, Di-hydroxy Alkenyl and | No |
| | Alkenyl-hydroxy | |
| Mouse | Hydroxy, Di-hydroxy and Alkenyl-hydroxy | No |

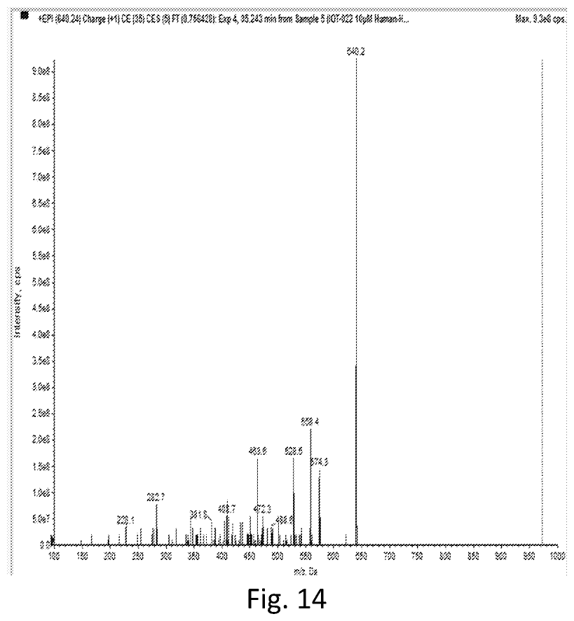

FIG. 14 shows the MS/MS spectra of a major metabolite formed from the incubation of the compound of Example 2 with human hepatocytes for 120 mins. The [M+H]⁺ ion and fragmentation pattern are consistent with oxidative metabolism to give the corresponding hydroxy compound.

The incubation studies with microsomes (Example 8) and hepatocytes (Example 9) show that the compounds tested are not metabolised via acyl-glucuronidation but rather via cyp oxidation only. This is a different metabolic profile than a corresponding carboxylic acid compound which are typically metabolised to the acyl-glucuronide. Such metabolism can give rise to reactive metabolites that cause liver toxicity and drug induced liver injury (Shipkova M, Armstrong V W, Oellerich M, and Wieland E (2003) Acyl glucuronide drug metabolites: Toxicological and analytical implications. Ther Drug Monit 25: 1-16; Regan S, Maggs J, Hammond T, Lambert C, Williams D and Park B K (2010) Acyl glucuronides: the good, the bad and the ugly. Biopharm Drug Dispos 31: 367-395; Shipkova M, Armstrong V W, Oellerich M, and Wieland E (2003) Acyl glucuronide drug metabolites: Toxicological and analytical implications. Ther Drug Monit 25: 1-16).

Both classes of FXR agonists, derivatives of bile acids e.g., obeticholic acid and non-bile acids, commonly contain the carboxylic acid functionality and are metabolised via Phase 2 conjugation, such as acyl-glucuronidation, due to their inherent chemical properties (Center for Drug Evaluation and Research, Application Number: 207999Orig1s000, Pharmacology Review(s); Gege C, Hambruch E, Hambruch N, Kinzel O, Kremoser C. Nonsteroidal FXR Ligands: Current Status and Clinical Applications, Handb Exp Pharmacol. 2019 Jun. 14; Tully D C, Rucker P V, Chianelli D, Williams J, Vidal A, Alper P B, Mutnick D, Bursulaya B, Schmeits J, Wu X, Bao D, Zoll J, Kim Y, Groessl T, McNamara P, Seidel H M, Molteni V, Liu B, Phimister A, Joseph S B, Laffitte B., Discovery of_Tropifexor_(LJN452), a Highly Potent Non-bile Acid FXR Agonist for the Treatment of Cholestatic Liver Diseases and Nonalcoholic Steatohepatitis (NASH), J Med Chem. 2017 Dec. 28, 60(24), 9960-9973); Michael K. Badman, Jin Chen, Sachin Desai, Soniya Vaidya, Srikanth Neelakantham, Jie Zhang, Lu Gan, Kate Danis, Bryan Laffitte and Lloyd B. Klickstei, Safety, Tolerability, Pharmacokinetics, and Pharmacodynamics of the Novel Non-Bile Acid FXR Agonist Tropifexor (LJN452) in Healthy Volunteers, Clinical Pharmacology in Drug Development, 2019, Dec. 10).

It is understood that the examples and embodiments described herein are for illustrative purposes only and that various modifications or changes in light thereof will be suggested to persons skilled in the art and are to be included within the spirit and purview of this application and scope of the appended claims.

That which is claimed is:

1. A method of modulating a farnesoid X receptor (FXR), the method comprising administering to a subject a compound of Formula I having a structure of: 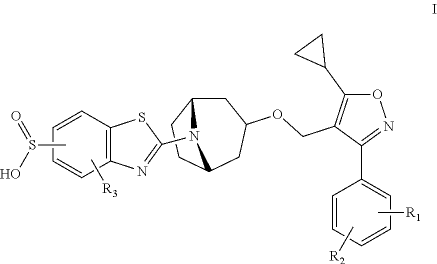
wherein:
R₁ and R₂ are independently selected from the group consisting of hydrogen, chloro, methyl, trifluoromethyl, methoxy, trifluromethoxy and cyclopropyl; and
R₃ is hydrogen, chloro, methyl, methoxy, fluoro or trifluoromethoxy;
or an enantiomer, stereoisomer, tautomer, solvate, hydrate, prodrug, amino acid conjugate, metabolite, or pharmaceutically acceptable salt thereof.

2. The method of claim 1, wherein the compound of Formula I has the structure of Formula II: 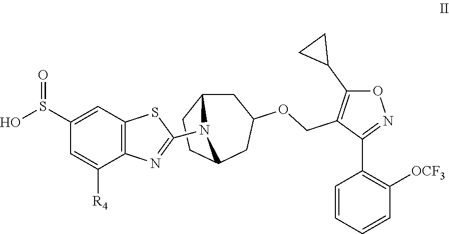
wherein:
R₄ is fluoro or methyl;
or an enantiomer, stereoisomer, tautomer, solvate, hydrate, prodrug, amino acid conjugate, metabolite, or pharmaceutically acceptable salt thereof.

3. The method of claim 1, wherein the compound of Formula I is selected from the group consisting of sodium 2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-sulfinate and sodium 2-((1R,3r,5S)-3-((5-cyclopropyl-3-(2-(trifluoromethoxy)phenyl)isoxazol-4-yl)methoxy)-8-azabicyclo[3.2.1]octan-8-yl)-4-methylbenzo[d]thiazole-6-sulfinate;
or an enantiomer, stereoisomer, tautomer, solvate, hydrate, prodrug, amino acid conjugate, metabolite, or pharmaceutically acceptable salt thereof.

4. The method of any one of claims 1-3, wherein the compound of Formula I upregulates and/or increases the expression of a FXR related gene, optionally wherein the compound upregulates a BSEP gene and/or a SHP gene.

5. The method of any one of claims 1-4, wherein the compound of Formula I reduces expression of a liver enzyme, optionally wherein the compound reduces expression of CYP7A1.

6. The method of any one of claims 1-5, wherein the compound of Formula I reduces liver triglycerides in the liver and/or plasma of a subject, optionally wherein the compound reduces liver triglycerides in the liver and/or plasma of the subject by at least about 5%, 10%, 15%, 20%, 25%, 30%, 35%, 40%, 45%, 50%, 55%, or 60% or more.

7. The method of any one of claims 1-6, wherein the compound of Formula I reduces cholesterol in the liver and/or plasma of a subject, optionally wherein the compound reduces cholesterol in the liver and/or plasma of the subject by at least about 5%, 10%, 15%, 20%, 25%, 30%, 35%, 40%, 45%, 50%, 55%, or 60%, or more.

8. The method of any one of claims 1-7, wherein the compound of Formula I is metabolised via a cyp oxidation pathway upon administration to a subject.

9. The method of any one of claims 1-8, wherein the compound of Formula I is not metabolised via acyl-glucuronidation upon administration to a subject.

10. The method of any of claims 1-9, wherein the compound of Formula I is present in a pharmaceutical composition comprising the compound of Formula I and a pharmaceutically acceptable carrier.

11. A method of activating a farnesoid X receptor (FXR), the method comprising administering to a subject a compound of Formula I having a structure of: 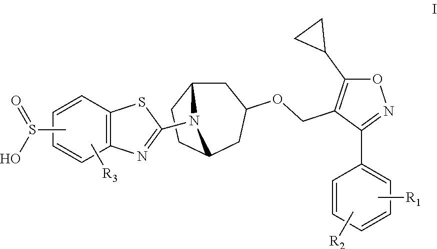
wherein:
R₁ and R₂ are independently selected from the group consisting of hydrogen, chloro, methyl, trifluoromethyl, methoxy, trifluromethoxy and cyclopropyl; and
R₃ is hydrogen, chloro, methyl, methoxy, fluoro or trifluoromethoxy;
or an enantiomer, stereoisomer, tautomer, solvate, hydrate, prodrug, amino acid conjugate, metabolite, or pharmaceutically acceptable salt thereof.

12. A method of treating and/or preventing a disease or disorder in which a farnesoid X receptor (FXR) plays a role, the method comprising: administering to a subject a compound of Formula I having a structure of: 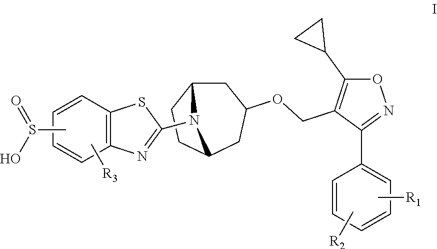
wherein:
R₁ and R₂ are independently selected from the group consisting of hydrogen, chloro, methyl, trifluoromethyl, methoxy, trifluromethoxy and cyclopropyl; and
R₃ is hydrogen, chloro, methyl, methoxy, fluoro or trifluoromethoxy;
or an enantiomer, stereoisomer, tautomer, solvate, hydrate, prodrug, amino acid conjugate, metabolite, or pharmaceutically acceptable salt thereof.

13. The method of claim 12, wherein the disease or disorder is a bile acid related disorder, metabolic syndrome, type-2 diabetes, diabetic nephropathy, hyperlipidemia, hypertriglyceridemia, obesity, liver cirrhosis, liver fibrosis, primary biliary cirrhosis (PBC), primary sclerosing cholangitis (PSC), fatty liver disease, nonalcoholic steatohepatitis (NASH), nonalcoholic fatty liver disease (NAFLD), alcoholic liver disease, chemotherapy associated steatohepatitis (CASH), hepatitis B, inflammatory autoimmune diseases, inflammatory bowel disease, Crohn's disease, ulcerative colitis, proctitis, pouchitis, Celiac's Disease, bile acid diarrhea, multiple sclerosis, atherosclerosis, kidney disorders (including chronic kidney disease), kidney fibrosis, lung fibrosis, cancer including hepatic cancers, colon cancers and breast cancers, and other disorders.

14. The method of claim 12, wherein the disease or disorder is a bile acid related disorder, fatty liver disease, nonalcoholic steatohepatitis (NASH) or nonalcoholic fatty liver disease (NAFLD).

15. A method of upregulating and/or increasing the expression of a FXR related gene in a subject, the method comprising: administering to a subject a compound of Formula I having a structure of: 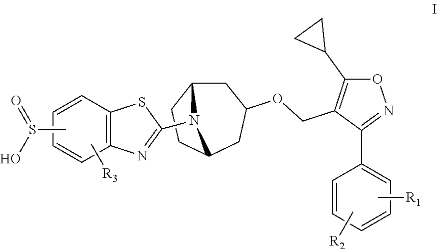
wherein:
R₁ and R₂ are independently selected from the group consisting of hydrogen, chloro, methyl, trifluoromethyl, methoxy, trifluromethoxy and cyclopropyl; and
R₃ is hydrogen, chloro, methyl, methoxy, fluoro or trifluoromethoxy;
or an enantiomer, stereoisomer, tautomer, solvate, hydrate, prodrug, amino acid conjugate, metabolite, or pharmaceutically acceptable salt thereof.

16. The method of claim 15, wherein expression of a BSEP gene and/or a SHP gene is upregulated and/or increased in the subject.

17. A method of reducing the expression of a liver enzyme in a subject, the method comprising: administering to a subject a compound of Formula I having a structure of: 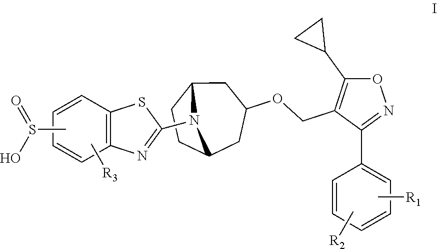
wherein:
R₁ and R₂ are independently selected from the group consisting of hydrogen, chloro, methyl, trifluoromethyl, methoxy, trifluromethoxy and cyclopropyl; and
R₃ is hydrogen, chloro, methyl, methoxy, fluoro or trifluoromethoxy;
or an enantiomer, stereoisomer, tautomer, solvate, hydrate, prodrug, amino acid conjugate, metabolite, or pharmaceutically acceptable salt thereof.

18. The method of claim 17, wherein CYP7A1 expression is reduced in the subject.

19. A method of reducing liver triglycerides in the liver and/or plasma of a subject, the method comprising: administering to a subject a compound of Formula I having a structure of 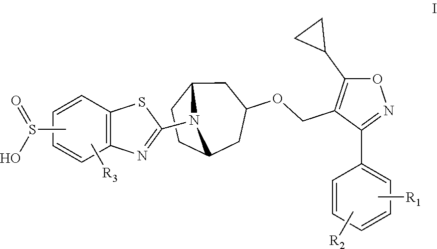
wherein:
R₁ and R₂ are independently selected from the group consisting of hydrogen, chloro, methyl, trifluoromethyl, methoxy, trifluromethoxy and cyclopropyl; and
R₃ is hydrogen, chloro, methyl, methoxy, fluoro or trifluoromethoxy;
or an enantiomer, stereoisomer, tautomer, solvate, hydrate, prodrug, amino acid conjugate, metabolite, or pharmaceutically acceptable salt thereof.

20. The method of claim 19, wherein liver triglycerides in the liver and/or plasma of the subject are reduced by at least about 5%, 10%, 15%, 20%, 25%, 30%, 35%, 40%, 45%, 50%, 55%, or 60% compared to the level and/or amount of liver triglycerides in the liver and/or plasma of the subject in the absence of a method of the present invention (e.g., prior to the administering step).

21. A method of reducing cholesterol in the liver and/or plasma of a subject, the method comprising: administering to a subject a compound of Formula I having a structure of: 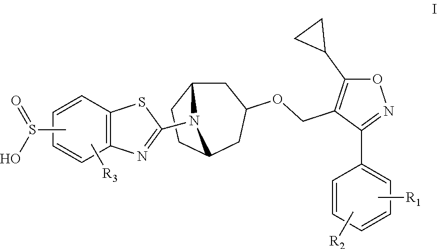
wherein:
R₁ and R₂ are independently selected from the group consisting of hydrogen, chloro, methyl, trifluoromethyl, methoxy, trifluromethoxy and cyclopropyl; and
R₃ is hydrogen, chloro, methyl, methoxy, fluoro or trifluoromethoxy;
or an enantiomer, stereoisomer, tautomer, solvate, hydrate, prodrug, amino acid conjugate, metabolite, or pharmaceutically acceptable salt thereof.

22. The method of claim 21, wherein cholesterol in the liver and/or plasma of the subject are reduced by at least about 5%, 10%, 15%, 20%, 25%, 30%, 35%, 40%, 45%, 50%, 55%, or 60% compared to the level and/or amount of cholesterol in the liver and/or plasma of the subject in the absence of a method of the present invention (e.g., prior to the administering step).

23. The method of any one of claims 11-22, wherein the compound of Formula I has the structure of Formula II: 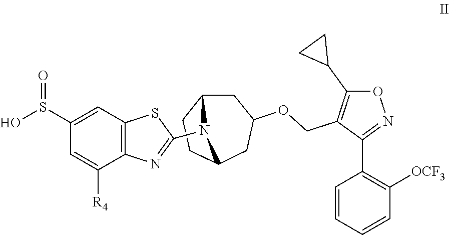
wherein:
R₄ is fluoro or methyl;
or an enantiomer, stereoisomer, tautomer, solvate, hydrate, prodrug, amino acid conjugate, metabolite, or pharmaceutically acceptable salt thereof.

24. The method of any one of claims 11-22, wherein the compound of Formula I is selected from the group consisting of sodium 2-(3-((5-cyclopropyl-3-(2-(trifluoromethoxy)phenyl)isoxazol-4-yl)methoxy)-8-azabicyclo[3.2.1]octan-8-yl)-4-fluorobenzo[d]thiazole-6-sulfinate and sodium 2-((1R,3r,5S)-3-((5-cyclopropyl-3-(2-(trifluoromethoxy)phenyl)isoxazol-4-yl)methoxy)-8-azabicyclo[3.2.1]octan-8-yl)-4-methylbenzo[d]thiazole-6-sulfinate;
or an enantiomer, stereoisomer, tautomer, solvate, hydrate, prodrug, amino acid conjugate, metabolite, or pharmaceutically acceptable salt thereof.

25. The method of any of claims 11-24, wherein the compound of Formula I is present in a pharmaceutical composition comprising the compound of Formula I and a pharmaceutically acceptable carrier.

26. The method of any one of claims 11-25, wherein the compound of Formula I is metabolised via a cyp oxidation pathway upon administration to the subject.

27. The method of any one of claims 11-26, wherein the compound of Formula I is not metabolised via acyl-glucuronidation upon administration to the subject.

28. The method of any one of claims 1-27, wherein the method reduces steatosis, hepatocellular ballooning, and/or lobular inflammation in the subject, optionally compared to steatosis, hepatocellular ballooning, and/or lobular inflammation in the subject prior to administering the compound of Formula I.

29. The method of any one of claims 1-28, wherein the method reduces the subject's NAFLD activity score (NAS), optionally compared to the subject's NAS prior to administering the compound of Formula I.

30. The method of any one of claims 1-29, wherein the method reduces fibrosis in the subject, optionally compared to fibrosis in the subject prior to administering the compound of Formula I.

31. The method of any one of claims 1-30, wherein the compound of Formula I is administered to the subject in an amount of about 0.05 to about 5 mg of the compound per kg of the subject.

32. The method of any one of claims 1-31, wherein the compound of Formula I is administered to the subject at least once a day.
